(19) United States
(12) Patent Application Publication
(10) Pub. No.: US 2009/0311682 A1
(43) Pub. Date: Dec. 17, 2009

(54) COMBINATION OF AVIAN CELL MARKERS

(76) Inventors: Bertrand Pain, Clermont-Ferrand (FR); Fabrice Lavial, Saint Etienne (FR); Elodie Bachelard, Marcy L'Etoile (FR); Guillaume Montillet, Villette D'Anthon (FR); Jacques Samarut, Lyon (FR)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/294,033

(22) PCT Filed: Mar. 19, 2007
(86) PCT No.: PCT/EP2007/052574

§ 371 Date: Jan. 27, 2009

(30) Foreign Application Priority Data
Mar. 24, 2006 (FR) 06 2592

(51) Int. Cl. C12Q 1/68; C40B 40/08

(52) U.S. Cl. 435/6; 506/17

(57) ABSTRACT

The present invention relates to a novel combination of avian cell markers which make it possible to characterize the said cells according to their phenotype, whether they are StX cells, stem cells or germ cells. The present invention also relates to a method for characterizing avian cells with the said markers, and to a method for culturing avian cells, in which the cells are characterized by the method according to the invention.

This invention relates to a new combination of avian cell markers allowing the characterization of said cells according to their phenotype, whether they are StX cells, stem cells or germ cells. This invention also relates to a process for characterizing avian cells with said markers, as well as a process of growth of avian cells, in which the cells are characterized by the process according to the invention.

The objective of the invention is to identify and use a single combinatorial of genes suitable for use as markers for defining, modifying, positively or negatively controlling the germinal competence of a stem cell of any embryonic, germ or adult type, in particular an avian stem cell.

A stem cell is a pluripotent or multipotent cell of embryonic or adult origin that has a capacity for self-renewal. In other words, a stem cell is a non-cancerous cell able to divide indefinitely in culture and produce a daughter cell having the same proliferation and differentiation capacities as the mother cell from which it originates.

Various classes of stem cells have been isolated according to their embryonic or adult origin and their somatic or germinal tissue origin. It has been shown that mouse embryonic stem cells (MESC) were able to contribute to the germ line when they are reintroduced into an embryo. The molecular mechanisms that control this germinal competence have not been fully identified.

Stem cells are cells that have the prodigious property to self-renew both in vivo, where they ensure maintenance and renewal of tissue, and in vitro, where they can be maintained with defined culture conditions.

Depending on their tissue origin and their differentiation potentiality, we can distinguish embryonic stem cells (ESC), pluripotent cells of embryonic origin, germinal stem cells (EGC) and somatic or adult stem cells of tissue origin (ASC). The last ones have a certain plasticity in their differentiation potentiality both in vitro and in vivo.

Embryonic stem cells (ESC)
MESC cells

Embryonic stem cells (ESC) cells were isolated and identified in the mouse in the 1980s (Martin, 1981; Evans & Kaufman, 1981) and their isolation has been described in numerous studies conducted heretofore with embryonic carcinoma cells (or EC cells) (for additional information, see Chambers & Smith, 2004). These mouse ESC cells (MESC) were obtained from by in vitro culture of mouse blastocysts 129/SV according to various protocols described (Robertson, 1987, Hogan et al., 1994). Mouse blastocysts before gastrulation have an internal cell mass or ICM of 50 to 100 cells, of which the molecular characterization is still in progress. The outcome of these cells is not identical in vivo and in vitro.

In vivo, epiblast cells will produce embryonic tissue and germ cells are formed in the proximal epiblast under the induction of the extra-embryonic ectoderm. The germ cells thus appear in a niche under the influence of various factors and cytokines. The complex molecular mechanisms responsible for this induction are only just beginning to be identified in the mouse (Saitou et al., 2003).

In vitro, the culture of blastocysts leads to their emergence, to adhesion and to the cell proliferation of the ICM, which, under suitable culture conditions, produces MESC cells. These in vitro culture conditions are now fairly well identified and standardized, in particular by the addition to the culture medium of at least one cytokine of the LIF family (Yoshida et al., 1994). New signaling pathways also appear to be involved in maintaining the pluripotent phenotype of MESC cells (Dani et al., 1998). Many studies have been published on the molecular mechanisms that control the pluripotency of MESC cells. In this model, various molecular and functional approaches have made it possible to identify the key role of genes oct-3/4 (Niwa et al., 2000; Niwa et al., 2002), nanog (Chambers et al., 2003; Mitsui et al., 2003) and stat3 (Niwa et al., 1998; Matsuda et al., 1999). A consensus diagram places the LIF and the BMP at the center of the mitotic signal and the genes Oct/nanog and stat3 as regulators and effectors of the proliferation-differentiation balance of the ES cells (Ying et al., 2003; Chambers et al., 2004). One of the downstream effectors is the proto-oncogene c-myc directly regulated by STAT3 (Cartwright et al., 2005). Other players are involved, such as the protein P53, which appears to control the expression level of nanog (Lin et al., 2005). The protein OCT-3/4 controls its transcription at the level of its own promoter in association with proteins of the HMG family, such as SOX-2 and/or SOX-3 (Okomura et al., 2005). In particular, the restriction of the expression of the gene Oct-3/4 at the mouse germ line is subject to the repressive action of the GCNF (Furhmann et al., 2001). The receptor lrh-1 is involved in maintaining an earlier stage of the development, as in epiblast cells (Gu et al., 2005) At least in the mouse, germinal competence therefore appears to require, inter alia, a high level of GCNF and a relatively low level of OCT-3/4, even if the expression of this gene oct-3/4 is essential to the germ line (Kehler et al.; 2004). The existence of numerous interactions between players of various signaling networks is a reality that is not yet fully understood for explaining the maintenance of the pluripotency of MESC cells.

It is noted that the success of the establishment of MESC cells also depends on the mouse stem used, with a facility for establishment from the genetic base 129SV. The molecular identification of this limitation has not been documented.

### PESC and HESC Cells

In primates, the first monkey ESC cells (PESC) were isolated by J. Thomson of the University of Wisconsin at Madison (Thomson et al., 1995) from blastocysts. Since then, other laboratories have isolated new monkey cell lines from various species. The characterization of these cells has also been the subject of numerous publications concerning both the biochemical characters (AP, telomerase, surface antigens, etc.) and the genes involved in maintaining pluripotency.

At the human level, ESC cells (HESC) were obtained in vitro by growth of blastocysts (Thomson et al., 1998). These cells have numerous similarities with MESC cells, but also major differences, such as sensitivities to various growth factors.

At the level of the mechanisms involved in maintaining the pluripotency of PESC and HESC cells, the genes identified in the mouse also appear to be involved, but the situation appears to be more complex. As an example, the sensitivity of PESC and HESC cells to LIF, and the activation of the Jak/stat pathway does not appear to be as critical as that of murine cells (Raz et al., 1999; Sumi et al., 2004; Humphrey et al., 2004). These cells are thus not exclusively dependent on this single cytokine, which makes the analysis of the proliferation signal more complex. Even if we observe the expression of the major players oct-3/4, nanog and stat3 as well as others such as rex1, foxD3, etc. in the primate ESC cells, the role of these genes would perhaps not be as decisive and the existence of alternative pathways is envisaged.

### CESC Cells

Chicken embryonic stem cells (CESC) were isolated by growth of chicken blastoderm cells at stage X (Pain et al., 1996; patent application N^{o} FR 94/12 598). These CESC cells have all of the characteristics of embryonic stem cells (ESC) including somatic and germinal colonization. Nevertheless, this germinal colonization appears to be difficult to maintain after several times in an in vitro culture, unlike the murine ES cells. In the collection and growth stage of the CES cells, the chicken embryo is considered to be a blastula already consisting of 50,000 to 60,000 cells, organized into two germ layers, the epiblast and the hypoblast. The only criteria of morphology, three-dimensional distribution and arrangement of cells do not make it possible to specifically identify various cell sub-types with certainty, in particular cells that have germinal competence (Petitte et al., 1990; Carsience et al., 1993; Thoraval et al., 1994). The detection of cells positive for the VASA protein in the very early chicken embryo (stage III-IV Eg&K) relaunched the hypothesis of the pre-formation of the germ line in the chicken. The cells would be individualized in the germ line upon the first divisions of the embryo (Tsukenawa et al., 2000) on a model closer to that of the fruit fly than the induction model observed in the mouse (Extavour et al., 2003; Saitou et al., 2002). These cells would then persist throughout the successive divisions until stage X.

For the chicken CESC cells, no data is available on the genes involved in maintaining the pluripotency and the emergence of the germ line in the chicken embryo. The expression profile of most of the genes identified in the other species as well as their regulation are completely unknown.

### Adult Stem Cells (ASC Cells)

Cells with properties of self-renewal and differentiation in vitro and in vivo were isolated from tissue and are called ASC (Adult Stem Cells) (Wagers and Weissman, 2004). These cells were isolated in various species, primarily mammals, from various tissues, in particular hematopoietic tissue (HSC for Hematopoietic Stem Cells) and nerve tissue (NSC for Neural Stem Cells). A full body of literature illustrates and seeks to characterize these cells (for additional information, see Shizuru et al., 2005, Mayhall et al., 2004). Many other cell types have been identified that have some of the properties of stem cells. The following can be cited:
mesenchymal cells (MSC) (Hamada et al., 2005)
muscle satellite cells (Charge and Rudnicki, 2004; Seale et al; 2004)
multipotent precursors ‘MAPC’ (Jiang et al., 2002)
mesangioblast cells (Cossu and Bianco, 2003; Minasi et al, 2002).

Their high differentiation plasticity observed makes it possible to consider adult stem cells as cells able to change their differentiation determinism according to the context, the site and the signals that they receive (Lakshmipathy, 2005, Galli et al., 2000). The relationship that exists, at least in vitro between an ESC cell and an ASC cell, may appear to be a continuum of limited differentiation steps. Indeed, it is possible to reproduce complete differentiation sequences in vitro from ESC cells in a given specific differentiation pathway. The control of these complex mechanisms for inducing differentiation pathways is of great importance in understanding the determinism of these cells.

### Germinal Stem Cells (Cellules EGC) and Germinal Competence

Unlike ESC cells, derived directly from an embryo obtained before the gastrulation stage, germinal embryo stem cells (EGC cells) are obtained from gonadal ridges that have been colonized by the germinal precursors. Over the course of the emergence of the germ line and depending on the species, the precursor cells are individualized in various territories of the embryo. The example most studied and which is beginning to be the one best deciphered at the molecular level is that of the mouse germ line.

Mouse EG cells (MEGC) have numerous similarities with MESC cells, but one of the essential differences concerns the epigenetic status of certain genes (Shiota et al., 2002, Reik et al. 2001, Tada et al., 1998). By epigenetic status, we mean the degree of direct modification of the DNA and associated proteins such as histones. This status determines, in large part, the transcriptional activity of the DNA which corresponds to the euchromatin or to heterochromatin domains which are transcriptionally inactive. Numerous molecules are involved in modifying and controlling this status, in particular certain methyltransferases such as dnmt genes, of which the effect on the ESC cells and during development is beginning to be (Gaudet et al., 2004, Biniszkiewicz et al., 2002, Hattori et al., 2002).

By germinal competence, we mean the capacity of a cell to produce a differentiated cell that is a precursor of a functional gamete. A primordial germ cell thus produces cells of the male line (spermatogonia) or of the female line (ovocytes), able to produce, respectively, mature spermatozoids and mature ovocytes.

This germinal competence can be observed in vivo or in vitro.

In vivo, depending on the species, germ cells are individualized according to a process of induction by the tissue and the surrounding cells in a niche. The examples most studied are those of the fruit fly D. melanogaster and the roundworm C. elegans, a species for which numerous mutants have been obtained. This process is beginning to be described at the molecular level in the mouse (Saitou et al., 2002, Ohinata et al., 2005) for which a first induction model has been proposed. It involves the notion of a specific niche in which germ cells are individualized. The induction is controlled by the surrounding cells via the factors BMP-4 and BMP-8, which modify the transcriptional level of key genes such as oct-3/4, blimp-1, the markers stella, fragilis, and so on.

In other species, the individualization of the germ line would have a strong maternal component. The RNA binding proteins would then play an essential role in the establishment of a gradient of certain components (proteins and RNA) in the unsegmented embryo, which would thus induce a “sequestration” of these proteins and/or messenger RNA able to induce the identity of germ cells in a defined location of the embryo. The genes involved in this compartmentalization are beginning to be particularly studied in the model organisms such as D. melanogaster and C. elegans (Extavour et al., 2003, Blackwell, 2004). The description, the number and the involvement of the various genes in these determinisms are documented in particular in scientific journals and various accessible sites such as http://germonline.igh.cnrs.fr/index.php.

In vitro, recent examples show that it is possible to obtain, to partially reproduce the conditions for differentiation of mouse embryo stem cells (MESC) into competent germinal cells. This process is experimentally dependent on growth factors and specific three-dimensional conditions. The factors BMP-4 and BMP-8, as well as hormones such as estrogens and FSH are found for obtaining germ cells from MESC (Hubner et al., 2003; Geijsen et al., 2004).

Various processes are known for growth of avian cells, stage X blastoderm cells (StX), stem cells or germ cells, in particular for virus replication or for the production of heterolog proteins and, as the case may be, the production of vaccines. Such processes are in particular described in the following patent applications and patents: WO 03/043415, WO2005/007840, WO 03/076601, WO 01/85938, WO 96/12793, EP 1149899, US 2002-192815 or U.S. Pat. No. 6,500,668.

Various means for differentiating the different cell forms above are known. It is nevertheless important to have techniques for characterizing phenotypes of different cells that are easier to use and of greater relevance.

Genes have been identified as being expressed either in chicken embryo stem cells (CESC) or in chicken germ stem cells (CEGC). The comparison of the relative expression levels of these different players makes it possible to propose a specific and original combinatorial that defines the molecular profile of each of these cells, as well as the necessary modifications for obtaining a certain germinal competence of the CESC cells.

The combination of markers and the process according to the invention make it possible in particular to ensure better traceability of the avian cell lines, but also to ensure good stability over time of the cell lines cultivated.
By stem cell, we mean any cell able to self-renew in vitro and able to produce specialized differentiated cells.
By germ cell, we mean any cell able to produce precursors or differentiated gametes of the male or female line.
By stage X blastoderm cell, also called “StX cell”, we mean blastoderm cells obtained by dissociation of the embryo from a fertilized egg just laid and of which the development stage corresponds to a stage X according to the table EG &K (Eyal Giladi & Kovak, 1976).
By DNA chip, we mean a set of genes, gene fragment or oligonucleotides deposited on a support (glass slide, nylon membrane, etc.) with a high density.
By meiosis, we mean the biological process that causes a diploid cell to reduce its gene pool to a haploid level. This process is accompanied by at least one cell division.
By functional gamete, we mean a haploid cell able to fuse with or provide a set of chromosomes to another haploid cell in order to form, in the case of fertilization, an egg, a diploid cell able to develop into an embryo.

The invention therefore relates to a combination of markers allowing the characterization of avian cells of StX-type, stem cells or germ cells, including:

a. at least one marker of a target gene preferentially expressed in StX cells chosen from the following genes: 1P06, 2contig58, 60S-L14, ATM, Bloom syndrome, BTEB4, CD9, CHD helicase, Clock, cwf16 (FLJ10374), CXCR4, Dnmt2, enxl (ho-zeste 2), eomes, EWS, FGF-4, GATA-5, HOJ-1, N-AGN6P deacetylase, N-Cor1, NF2, p53, pml, rbm6, SA-2, SA-3, SARA, SCYE1, SEF, sf-1, SnoN, SOCS13, SSB-1, TC87479, T-cell APP 2C, TGF-beta2, WD40/FYVE-d protein 2, WD-RP3, Zan75, ZPC and combinations thereof, and/or

b. at least one marker of a target gene preferentially expressed in stem cells chosen from the following genes: 1P06, 1P08-A09, activin RIIB, astacin, Claudin-3, dapper-1, Dorfin, FPP synthase (fps), GalNAc-T3, gcnf, HSPb7, IRX4, LMX, pax-6, Slc38a2, sox-3, tra1 gp96, wnt-10a, wnt-11 and combinations thereof, and/or

c. at least one marker of a target gene preferentially expressed in germ cells chosen from the following genes: adiponectin, BMP-2IK, bruno like, CD34, CDK5 activator 1, dkk1, dkk3, DMRT1, emx2, endoglin, FAST-1, FGF R, FLJ00188, flk-1, gata-4, gcl, LHX9, NOS type III, plzf, PRL-R box1l, PTEN, SAMSN-1, slug, smad3, Smarcd3, sox-9, Strat8, TACC2, TC95408, TC97694, TGF RII, TGF RIII, tie2, tie-2, TR-alpha, VE-Cadherin, vera, Wisp-1 and combinations thereof.

A list of the target genes preferentially expressed in StX cells, stem cells and germ cells is provided in FIGS. 1 to 3 respectively with the primers used to isolate them by PCR.

It is understood that the combination according to the invention includes at least two markers chosen either from the same group or from two distinct groups defined above (markers of genes expressed in StX cells, stem cells or germ cells).

Preferably, the marker of a gene preferentially expressed in StX cells is chosen from the markers of the following genes: 1P06, ATM, CXCR4, eomes, FGF-4, GATA-5, NF2, SOCS13, SSB-1, TC87479, T-cell APP 2C, TGF-beta2, WD-RP3, ZPC and combinations thereof. More preferentially, the combination according to the invention includes at least one marker of the ZPC gene.

According to another preferential embodiment of the invention, the marker of a gene preferentially expressed in stem cells is chosen from the markers of the following genes: 1P06, activin RIIB, astacin, Claudin-3, dapper-1, FPP synthase (fps), GalNAc-T3, gcnf, LMX, pax-6, tra1 gp96, wnt-10a and combinations thereof. More preferentially, the combination according to the invention includes at least one marker of the 1P06 and tra-1 genes.

According to yet another embodiment of the invention, the marker of a gene preferentially expressed in germ cells is chosen from the markers of the following genes: adiponectin, DMRT1, endoglin, FAST-1, FGF R, FLJ0188, gata-4, LHX9, plzf, PRL-R boxll, PTEN, Strat8, TGF RIII, Wisp-1 and combinations thereof. More preferentially, the combination includes at least one marker of the dmrt-1 and vasa genes.

According to the preferred embodiment of the invention, the combination includes the combination of markers of the following genes: 1P06, tra-1, FPP synthase, astacin, GalnacT3, lmx, gcnf, eomes, id2, FGF-4, ZPC, gata-5, dmrt-1, lrh-1, and pten.

By “marker”, we mean, according to the invention, any biological, chemical or physical means allowing the identification, and as the case may be the quantification, of the expression of a target gene in a cell, an avian cell according to the invention. Such markers are well known to a person skilled in the art. Their composition will depend in particular on the target gene and the method for detecting the expression of said target gene. In particular, we will cite the methods using the antibody/antigen pairs, with the antibody binding specifically to the antigen, which is constituted by the product of the expression of a gene identified above, mRNA or cDNA or polypeptide, or a fragment of these. We will also cite the nucleic acid fragments able to hybridize specifically with the mRNA expressed by said genes or the corresponding cDNA, or fragments of these.

The various types of markers can of course be combined in the combination according to the invention.

Advantageously, the markers according to the invention are nucleic acid sequences able to hybridize with RNA or cDNA, products of the expression of the target genes identified above.

Among these nucleic acid sequences, we can cite the specific primer sequences identified in FIGS. 1 to 3.

According to a more specific embodiment of the invention, the combination of markers is assembled on a single support, preferably a standard support. A person skilled in the art knows these various supports, of which the sizes vary according to the type of markers and apparatuses that may be used to detect the expression of the target gene(s).

Advantageously, the combination of markers according to the invention is in the form of a DNA matrix, including a support on which the nucleic acid fragments able to hybridize with the target genes are arranged. The sizes of such supports may vary according to the preparation and detection technologies used. Such supports of reduced sizes are also called DNA chips.

Advantageously, the combination of markers according to the invention is arranged on a DNA chip. The DNA chip including the combination of markers according to the invention is also a part of the invention.

According to a specific embodiment of the invention, the combination of markers can also include at least one marker of a gene that is expressed in two groups of cells: StX cells and stem cells, stem cells and germ cells, or germ cells and StX cells.

Such genes are identified in FIGS. 4 to 6, respectively, with the corresponding primers.

The means for detecting “expression product”/marker interactions are well known to a person skilled in the art. Numerous references of scientific publications on the preparation and use of DNA chips, as they concern academic research institutions or corporations selling the means for preparing and using DNA chips (reading apparatuses, signal processing software, databases, etc.) are provided in particular on the websites: www.gene-chips.com, www.deathstramic.com/science/biology/chips.html or http://cmgm.stanford.edu/pbrown/mguide/index.html, of which the content and that of the references cited is incorporated here by reference.

This invention also relates to a process for characterizing an avian cell including the analysis of the expression of genes expressed in said cell, using a combination of markers as defined above and in the examples, and the characterization of the phenotype of the analyzed cells. The specific methods of gene expression analysis will depend on the markers used in the combination according to the invention defined above and in the examples.

The invention also relates to a process of growth of avian cells including the culture of cells in a suitable culture medium and the characterization of said cells using a process according to the invention, with a combination of markers according to the invention as defined above and in the examples. Advantageously, the culture process according to the invention also includes a step of isolating cells selected from StX cells, stem cells and germ cells.

The invention also relates to a process of growth of avian cells in which germ cells are obtained from StX cells. The process according to the invention is distinguished from the processes of the prior art that do not enable such a transformation in that StX cells are cultivated in a suitable culture medium without the addition of inactivated “feeder” layer.

The inventors were able to observe that, while the usual practice of adding “feeder” is necessary to obtain stem cells, by not adding such a “feeder”, it was possible to obtain, in an entirely unexpected manner, germ cells.

The characteristics of the “feeder” are well known to a person skilled in the art, described in particular in Robertson et al., 1987, Karagenc et al., 2000 of which the content is incorporated here by reference.

This characterization of germ cells and not embryonic stem cells was achieved by implementing the characterization process according to the invention, with a combination of markers according to the invention.

Other features of the invention will appear on reading the examples provided below.

### BRIEF DESCRIPTION OF THE FIGURES

FIG. 1: Table of genes expressed in StX cells.

FIG. 2: Table of genes expressed in stem cells.

FIG. 3: Table of genes expressed in germ cells.

FIG. 4: Table of genes expressed in StX cells and stem cells.

FIG. 5: Table of genes expressed in stem cells and germ cells.

FIG. 6: Table of genes expressed in germ cells and stage X cells.

FIG. 7: Growth curve of GF58 cells.

FIG. 8: Expression level of several genes in the GF58 cells (p7).

FIG. 9: Analysis of the expression of genes in the p3g and p4g fractions with respect to the level observed in the gonads (=1). A ratio >1 suggests that the gene is expressed more in the fraction tested than in the entire embryonic gonad, and the reverse.

FIG. 10: Distribution by class of genes tested.

FIG. 11: Variation in the expression level of genes of class 1 during induction of the differentiation.

FIG. 12: Variation in the expression level of genes of class 2 during induction of the differentiation.

FIG. 13: Variation in the expression level of genes of class 3 during induction of the differentiation.

FIG. 14: Comparison of the expression profiles between the stage X blastoderm cells, the CESC cells and the CEGC cells.

FIG. 15: Comparison of the expression profiles between the CESC cells, the stage X blastoderm cells and the CEGC cells.

FIG. 16: Comparison of the expression profiles between the CEGC cellules, the stage X blastoderm cells and the CESC cells.

FIG. 17: Protein and cDNA sequence of the chicken nanog gene.

FIG. 18: Protein and cDNA sequence of the chicken gcnf gene.

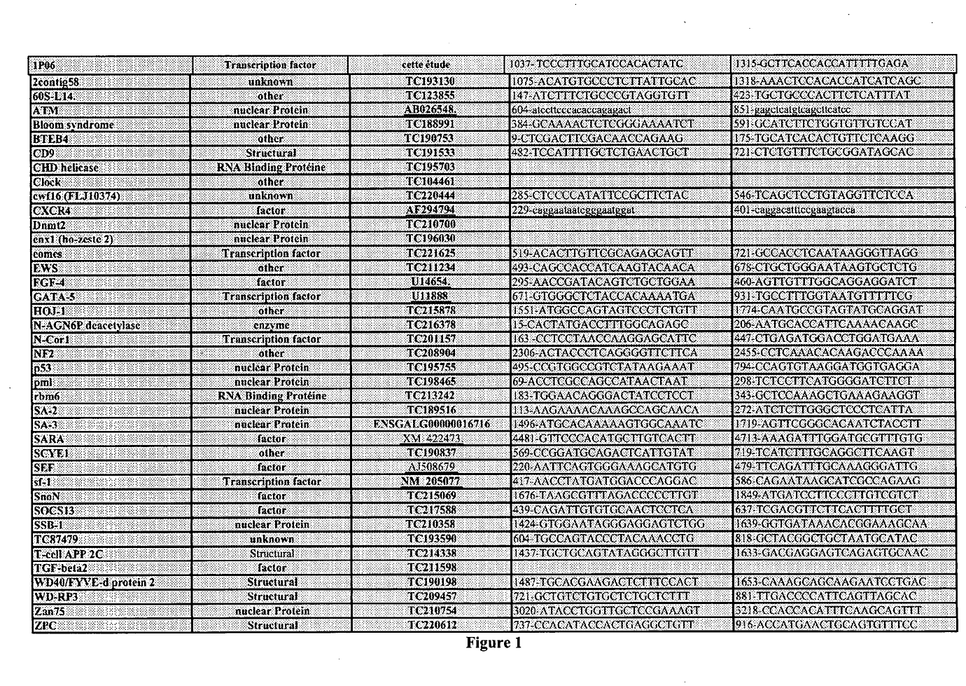

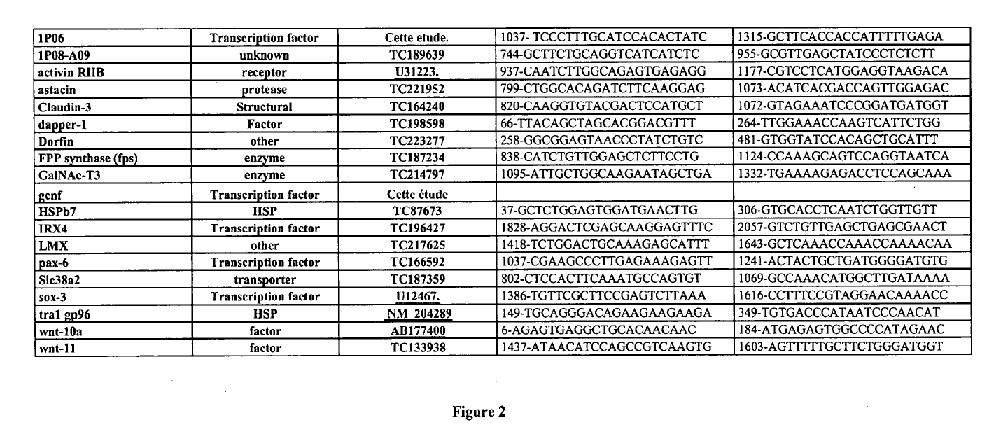

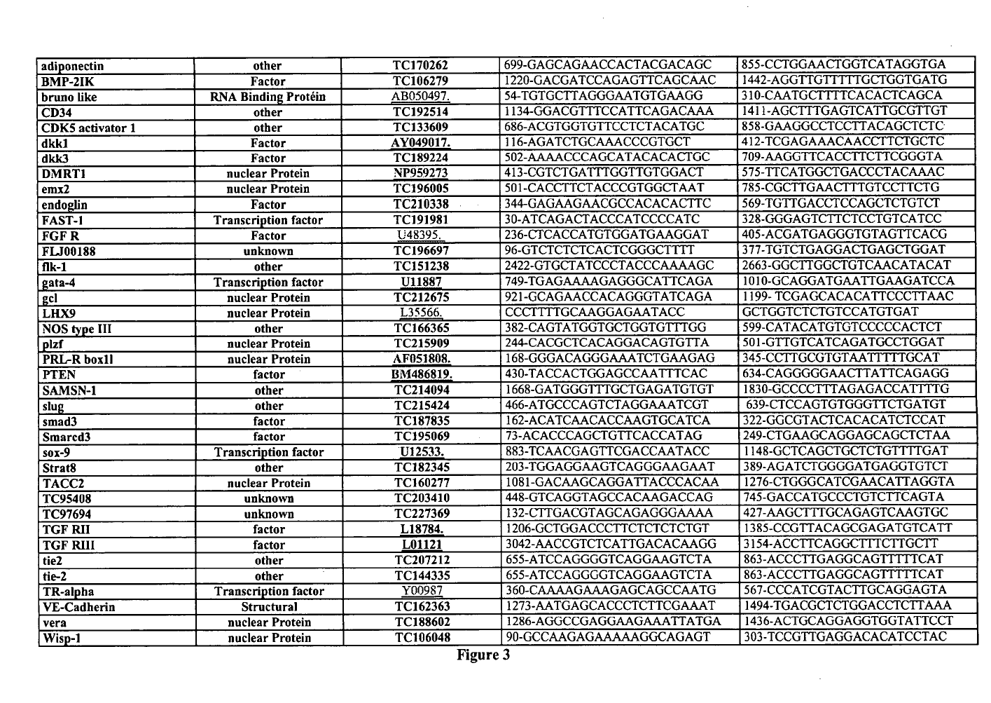

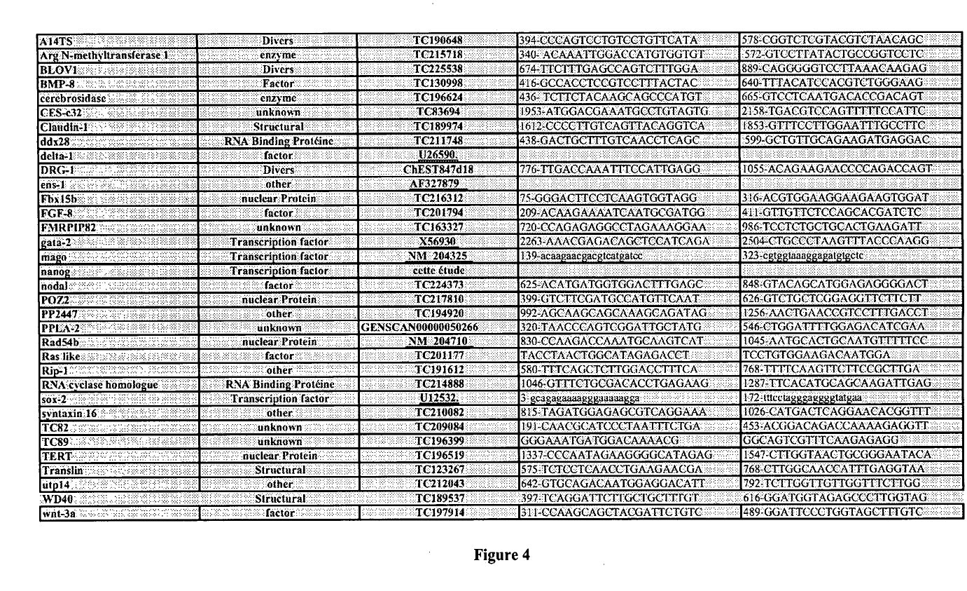

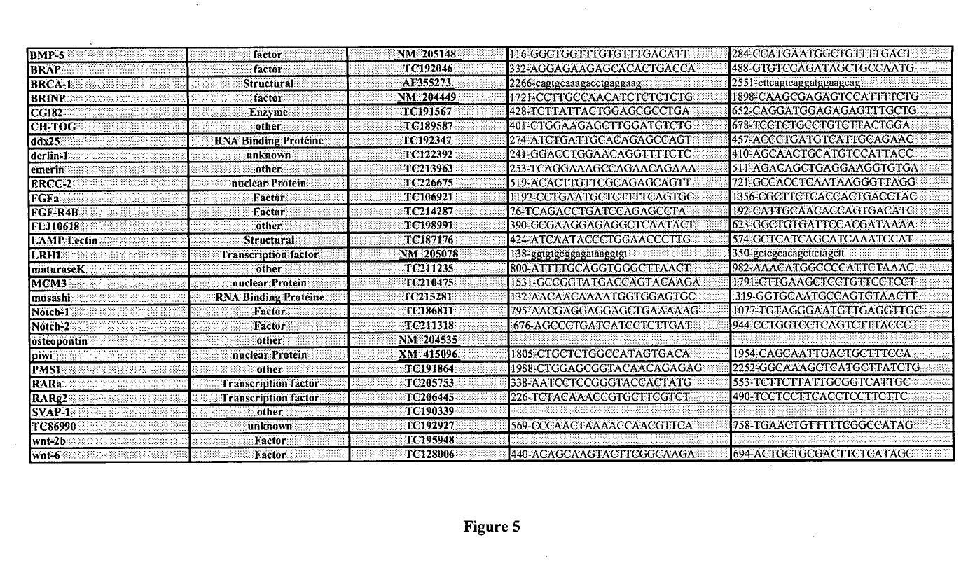

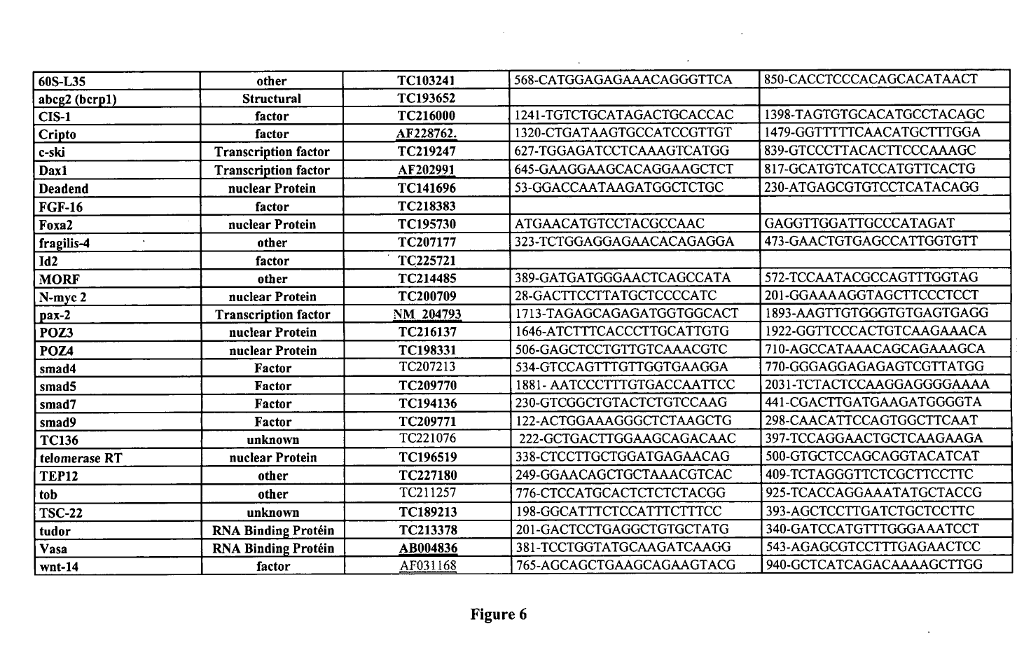

### EXAMPLES OF EMBODIMENTS

### Example 1

### Obtaining Embryonic Stem Cells in Vitro

Chicken embryonic stem cells are obtained in vitro from embryonated eggs. Two approaches are used to obtain populations of embryonic stem cells proliferating in vitro.

In a first aspect, the cells are obtained under conditions similar to those previously described (Pain et al., 1996). Briefly, the non-incubated embryonated eggs are cleaned and broken. The yolk is separated from the white and the embryo is collected by a Pasteur pipette or a paper ring as described by Carscience et al., 1993. The cells thus obtained are deposited on an inactivated feeding layer in a proliferation medium containing the elements essential for cell proliferation such as fetal bovine serum, amino acids and cytokines and growth factors.

In another aspect of the invention, the cells are seeded directly in an untreated plastic cell culture box intended for bacteriological culture use. In this last case, the cells are lightly agitated. Aggregates of stem cells that proliferate after several days in suspension are formed.

After several days, the embryonic stem cells form small compact masses that are dissociated using proteolytic enzymes and are seeded in new culture boxes for in vitro amplification.

After several passages, the culture becomes more homogeneous and the predominant cell type is the chicken embryonic stem cell (CESC). These CESC cells are characterized by the various markers described above, in particular the presence of the alkaline phosphatase activity, the endogenous telomerase activity and their reactivity with respect to specific antibodies (Pain et al., 1996).

To characterize the CESC cells obtained in culture, an electron microscopy observation was performed. It shows that the isolated cells have many microvilli at their surface, which had never been presented or described for a CESC cell; that they disappear as soon as the cells are in a mass or interact with other homologous or heterologous cells (feeder cells); that the various structures and ultrastructures recognized by EM indicate a significant metabolism (ribosomes, reticulum, numerous mitochondria), high transcriptional activity by the presence of a large nucleolus; that these CESC cells have sizes of 7 to 15 mm on average with a relatively homogeneous substantially spherical core with an average size of 4 to 8 mm. The intracellular space of the cells in mass is very limited, because the cells are especially connected.

### Example 2

### Obtaining Germ Cells in Vitro

In the bird, the molecular players in the determinism of germ cells have not yet been clearly identified. This determinism remains a relatively undocumented process. The stage of development of the embryo at time of laying is stage IX-XI according to the development table proposed by Eyal Giladi & Kovak (1976). At this stage, the embryo already contains 40,000 to 60,000 cells according to estimations and is composed of two germ layers, the epiblast and the hypoblast. At this stage, the presence of cells positive for the vasa gene (Tsukenawa et al., 2000) indicates that the germ cells are already determined. Various publications have described obtaining germ stem cells in vitro by growth of chicken embryo gonadal ridge cells. We can cite in particular the work of Ha et al., 2002; Park et al., 2003 based on the stage 28 embryo according to the development table proposed by Hamburger & Hamilton (1951) corresponding to around 5-6 days of development.

In another aspect of the invention, the embryo cells are collected as described in example 1, but are seeded in a culture box without a feeder layer. The culture box has been pre-treated for at least one hour or more with an egg white solution or another solution rich in serum protein, such as seric albumin. This white solution can be egg white directly collected or diluted with PBS. Preferably, a solution diluted between 5 and 10 times is used. The solution therefore contains the main components of the egg albumin, such as the ovalbumin, the conalbumin, the ovomucoids, the ovomucin and the lysozyme, as well as the other more minor components. A purified ovalbumin or conalbumin solution can also be used.

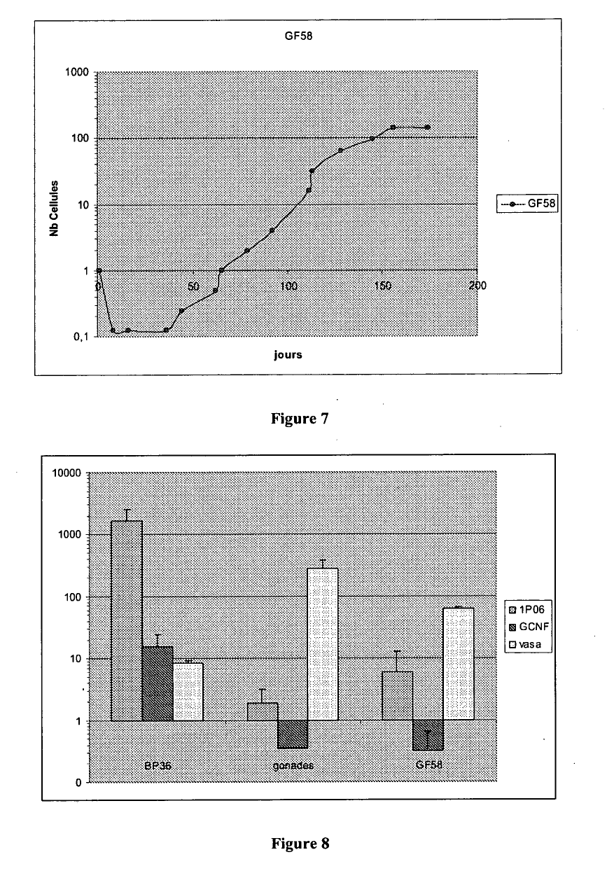

In one aspect of the invention, stage IX-XIII blastoderm cells are seeded in a culture box under culture conditions that promote the multiplication of “vasa” positive germ cells. Thus, the boxes have been treated with serum to produce a “coating” equivalent to an extracellular matrix intended to promote the adhesion and non-differentiation of cells, but without the addition of an inactivated “feeder” layer. After several days, cell foci appear, showing slow proliferation. This process persisted and the cells called GF58 continued to proliferate for at least 200 days. This proliferation, while particularly slow, is nevertheless continuous in the form of several compact cell foci. The passages are delicate, because it is difficult to dissociate the cells. A growth “curve” was produced (FIG. 7), based on the dilution coefficients during the cell passages, because it appeared to be impossible to count the cells individually in each (rare) passage.

These cells have a specific morphology and proliferate in small masses at the center of a focus of adherent and pavimentous cells, with a large core. The size of the foci increases very slowly.

Tests for placing the cells in the presence of “feeder” appeared to be catastrophic, as the cells do not resist this environment. Tests involving the addition of BMP-4, FGF-4 to the culture medium did not have negative effects on proliferation and did not cause any major modification. A slight stimulation was observed.

In passage 7 (after 79 days of culture), the cells have a high alkaline phosphatase activity and are positive for the antibodies SSEA-1 and EMA-1. In this same passage 7, several cells were collected for molecular analysis. The results (FIG. 8) show that the expression profile of the genes 1P06, gcnf and vasa is closer to that of the gonads than the CESC cells.

This very important result supports the idea of maintaining vasa positive germ cells after 80 days of culture, obtained by growth of stage X blastoderm cells. These cells are germ cells maintained in vitro.

### Example 3

### Obtaining Germ Cells by Embryonic Gonad Cell Sorting

To obtain purified germ cells in a quantity that can be used for molecular analysis, a FACS sorting approach is initiated. The approach is based on the presence of a membrane protein, the ABC receptor/Bcrp1/ABCG2 transporter, of which the expression was initially identified in hematopoietic stem cells. This molecular appears to have a physiological detoxification role and is responsible for the active Hoechst transport from the cell, when this drug is placed in the presence of the cell. This property appears to be specific to stem cells and is currently used to identify “SP” or “side population” cells according to their cell-sorting characteristic in various tissues (Zhou et al., 2001). Interestingly, the mouse germ cells appear to have this property (Lassalle et al., 2004).

In a first approach, the expression level of the bcrp1 messenger is higher in chicken germ cells than in CESC stem cells (cf. infra). This gene thus becomes an additional marker for identifying a stem cell in the chicken and a germ cell.

To test the role of this molecular as a marker in CESC cells and chicken germ cells, fractionation studies on chicken embryonic gonads were conducted. The embryonic gonads of male embryos at 15 to 21 days of development are collected after decapitation of the embryo and placed in iced. The gonads are placed in the complete HBSS 1× medium (GIBCO ref 14175-046) containing 20 mM Hepes pH 7.2, 1.2 mM MgSO4 7H₂O, 1.3 mM CaCl₂ 2H₂O, 6.6 mM sodium pyruvate, 0.05% lactate and 1% of the penicillin streptomcyin antibiotic mixture. The gonads are dissociated mechanically by successive passages in a 18G1/2 then a 20G1/2 needle until obtaining a single-cell suspension, which is then filtered on a 100-μM nylon membrane (Falcon 352360), counted by cell counting with trypan blue and centrifuged for 10 minutes at 400 g at 4° C. The pellet is placed in the complete HBSS medium.

A portion of the cells (from 10⁵ to 2×10⁶ cells) is subjected in complete HBSS to a verapamil treatment (V4629 SIGMA) or to the drug Ko143 (Lassalle et al., 2004, Allen et al. 2002) at respective concentrations of 75 μg/ml and finally 200 nM/ml. The cells are placed for 20 minutes in the incubator at 37.5° C. 7.5% CO₂. After this incubation, the cells are rinsed with PBS 1×, centrifuged for 10 minutes at 400 g and the pellet is placed in 1 ml of complete HBSS. The control cells not treated with verapamil or Ko143 are placed in ice.

The cells treated with verapamil, Ko143 or untreated are placed in complete HBSS at a concentration of 1 to 2.10⁶ cells/ml, then incubated with the Hoechst 33342 (ref. B2261, Sigma) at a final concentration of 5 μg/ml for 45 minutes in an incubator at 37.5° C. 7.5% CO₂. At the end of this incubation, the cells are rinsed with PBS 1×, centrifuged for 10 minutes at 400 g and the pellet is placed in 1 ml of complete HBSS with 0.5% SVF. This is all then placed in ice. Propidium iodide is added extemporaneously at a concentration of 2 μg/ml before the FACS analysis.

A FACS distribution profile shows the presence of various cell populations in the presence or absence of various drugs. It is thus possible to define two main populations, p3g (R1) and p4g (R2). The population called p3g is most sensitive to the action of drugs known to inhibit the abcg2 channel, in particular Ko143.

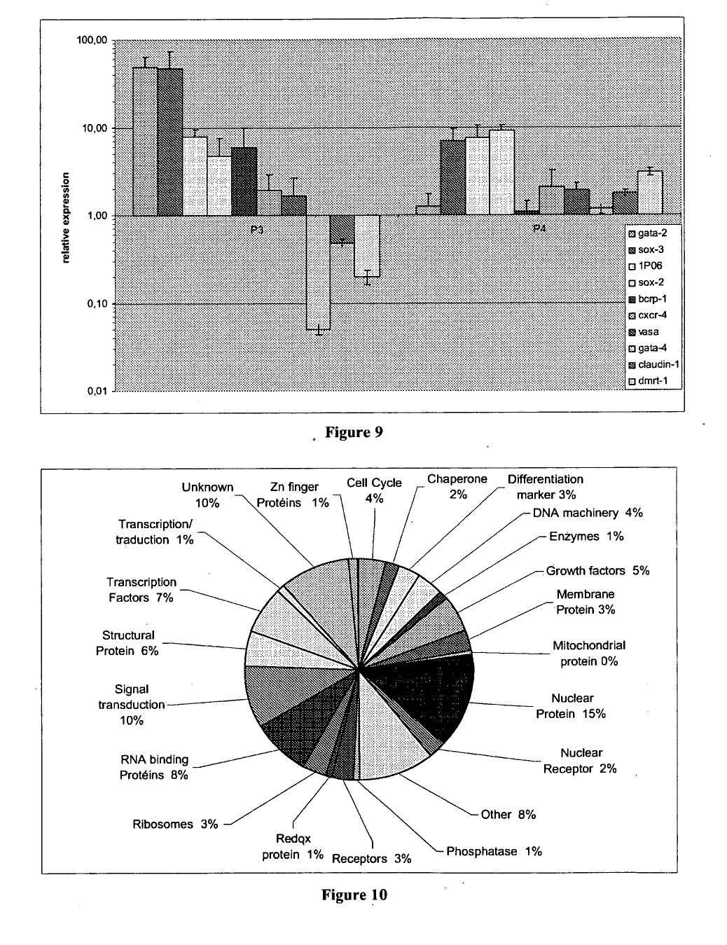

The p3g and p4g populations were sorted by FACS and the RNA of these cells were analyzed by Q-PCR (FIG. 9). On the few genes tested, it is possible to identify the p3g fraction as being enriched with germ cells, because this fraction is enriched with bcrp-1 (abcg2); by contrast, the p4g fraction is distinguished by an enrichment in dmrt-1 and gata-4, markers characteristic of support cells.

The detection of germ cell markers more highly expressed in this sub-population p3g confirms the interest of this enrichment method in obtaining purified germ cells.

### Example 4

### Identification of Genes Involved in the Pluripotency of Avian Embryonic Stem Cells (CESC)

To identify the genes involved in maintaining the pluripotency of the CES cells, various complementary screening strategies make it possible to characterize the transcriptional level of the CESC cells. Among the approaches, we can cite differential expression library screening, in silico data library screening, specific chicken DNA chip screening, and SAGE library screening. By using these various approaches, a list of candidate genes is defined. The expression level of these genes is tested by a semi-quantitative and quantitative PCR approach. The 800 different genes tested are distributed into different classes (FIG. 10).

The messengers (mRNA) are extracted from the proliferating CESC cellules using an RNA-Easy kit (Qiagen, Ref. 74104). At the end of the column preparation, the RNAs are collected in 50 ul of RNase free water, assayed and preserved in precipitate in ethanol at −80° C. A reverse transcription reaction with an oligodT primer is performed 3′ with the reverse transcriptase Superscript II (Invitrogen, Carlsbad) at 42° C. for 1 hour with 2 μg of total RNA from the undifferentiated proliferating CESC cells. The RT product is diluted 100 times in Millipore water and a quantitative PCR reaction is launched in a plate with the Mx3500P apparatus (Stratagene). The reaction mixture of 25 μl in water includes 2 μl of the diluted RT product, 12.5 μl of the Quantitect SYBRGreen Mix (Qiagen, ref. 204245), and 10 pM of each primer. After 15 minutes of denaturation, the mixture is subjected to 40 cycles consisting of 30 seconds of a denaturation phase at 95° C., 30 seconds of a hybridation phase at 55° C. and 30 seconds of an elongation phase at 72° C. Two or three independent tests are performed for each gene and the average is taken as the relative expression value.

To validate the data obtained by quantitative PCR, two complementary methods are used: the dissociation curve (melting curve) and the calculation of the efficacy of the PCR by the internal standard method. This last analysis makes it possible to evaluate whether each DNA molecule is indeed amplified specifically in each PCR cycle. The dissociation curve provides information on the number of amplicons of different types present in the tubes at the end of the amplification, and makes it possible to test the specificity of the primer pair used, for which a single amplicon must be formed. This curve also makes it possible to detect a possible contamination by the genomic DNA on the condition that the primers are chosen between two exons of a gene. This curve is produced at the end of 40 quantitative PCR cycles by a final cycle that includes denaturation for 1 minute at 95° C., hybridation for 1 minute at 55° C., then a temperature increase from 55° C. to 95° C. with a fluorescence reading every 0.2 seconds. Thus, at 55° C., the DNA fragments amplified during the 40 PCR cycles are hybridized and the fluorescence measured will then be at its maximum. By increasing the temperature very progressively, the temperature at which the two DNA strands separate is measured very precisely, which causes a drop in the fluorescence, as fluorochrome is incorporated only in bicatenary DNA. This temperature is a function of the size and basic composition of the amplicon. If a non-specific product is present, the drop in fluorescence will have two levels each corresponding to an amplicon. Only primer pairs and experiments with a single level are validate. Simultaneously, a reverse transcription (RT) reaction is performed on 2 ug of RNA so as to validate the efficacy of the PCR according to the conditions described above. A series dilution range of this RT product is obtained and arbitrary amounts are provided in the data tables proposed by the analysis software provided with the quantitative PCR apparatus (Stratagene, MXP 3500). For each dilution, a quantitative PCR is performed with the primer pair to be tested and the start of the exponential amplification of the amplicon is obtained at a specific cycle of the PCR. This Ct value obtained for each dilution makes it possible to obtain a line Y=aX+b where Y is the Ct value for a given point, X is the initial amount of material for each dilution and shows the slope of the line. The efficacy E of the PCR is then given by the formula E=10^{(1/-a)}. This efficacy must be between 1.8 and 2.2 so that the primer pair and the PCR conditions are validated. A value of 2 corresponds to a 100% amplification.

To enable a comparison of the variations in expression levels of various genes, it is necessary to compare these levels with that of a gene expressed constantly between the samples and the experimental conditions. The gene chosen for most of the experiments conducted is the RS17 gene (X07257), a gene coding for a ribosomal protein. It has indeed been shown that the expression level of this gene varied only slightly or not at all according to the state of the various cells studied. Thus, the differential observed between two Ct values indicates a modulation of the expression level of the gene of interest and is not due to a variation in the amount of starting material. The calculation of the relative level is given by the formula: D=2̂((CtX1−CtX2)/(CtR1−CtR2)) in which:

CtX1 represents the Ct of gene X for cell type 1

CtX2 represents the Ct of gene X for cell type 2

CtR1 represents the Ct of the control gene RS17 for cell type 1

CtR2 represents the Ct du of the control gene RS17 for cell type 2.

The relative expression level of genes with respect to their expression level in an embryonic fibroblast shows three categories of genes according to the intensity of the expression. It is possible to identify genes overexpressed at vary high levels and of which the relative expression level with respect to embryonic fibroblasts is greater than 100 times. These genes may be considered to be “specific” to CESC cells. The class of genes of which the relative expression level is between 10 and 100 currently includes around a dozen genes. Finally, it is possible to distinguish the class of genes of which the relative expression level is between 2 and 10 times that of embryonic fibroblasts.

### Example 5

### Monitoring of the Expression of Genes During CESC Cell Differentiation

Certain genes identified during the screening appear to have an expression specific to CESC cells. The expression differential between embryonic fibroblasts, CESC cells and CEGC cells indicate that the expression level of the genes varies according to the cell type. It also appears to be essential to characterize the variations in expression levels during differentiation of CESC cells.

Experiments on induction kinetics were performed, in particular by seeding cells in the differentiation medium without growth factors or cytokines in the presence or in the absence of an inducer such as retinoic acid at 10⁻⁷M. The stimulated cells are collected at different times and the expression levels of the genes are analyzed by QRT-PCR as described above (cf. example 4). The analysis of variations in the expression of certain genes shows different kinetics according to genes.

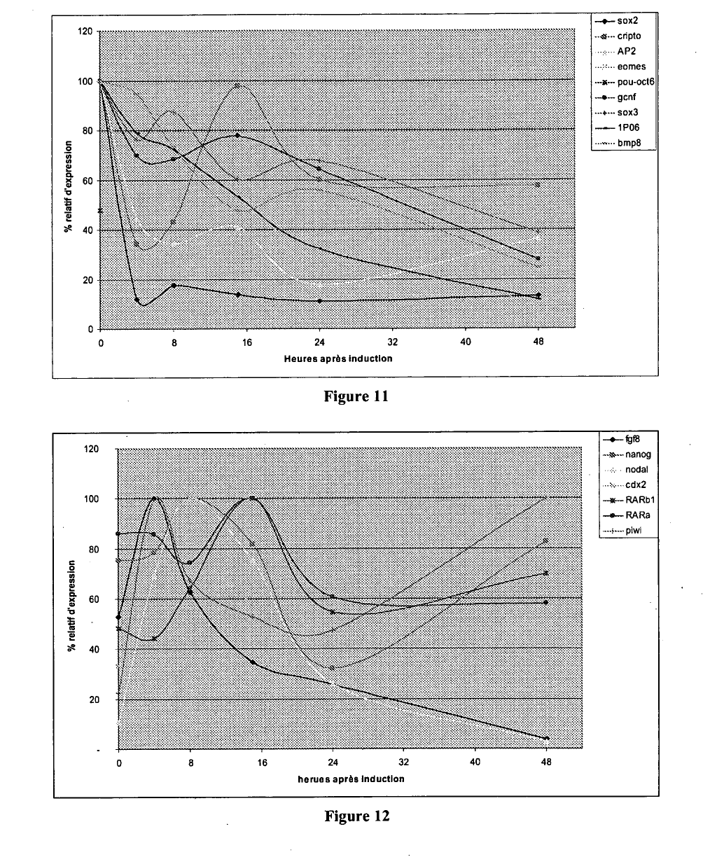

We can distinguish three major classes of genes according to the kinetics of variations in their expression level. In class 1, we find the genes for which the expression level is maximal before induction and that very quickly falls with the addition of retinoic acid to the culture medium (FIG. 11). This class includes the genes sox-2, cripto, AP, eomes, oct-6, gcnf, sox-3, 1P06, GTT3, RARg2, BMP-8, gata-4, and so on.

In class 2, we find the genes for which the optimal expression level is reached between 4 and 8 hours after inducing the differentiation (FIG. 12), such as the genes fFGF-8, nanog, nodal, cdx-2, RARb1, RARa, piwi, TERT, and so on.

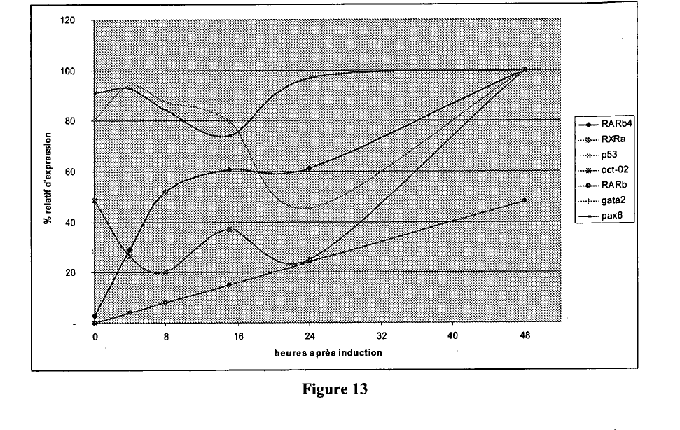

In class 3, we find the genes for which the maximum induction level is obtained starting at 24 hours after the start of the induction of the differentiation (FIG. 13), in particular, the genes piwi, p53, TERT, RXRa, Oct-2, RARb, gata-2, pax-6, and so on.

In these successions of variations and induction, one will also note the speed of variation of certain of these players, such as the genes cripto/.nodal and FGF-8/BMP-8 of which the variations are opposed.

### Example 6

### Identification of Genes Expressed in Stage IX-XII Blastoderm Cells

The stage IX to XII blastoderm cells are collected as described above (Pain et al., 1996 FR N^{o}) In regard to the presence of residual yolk, even after preparation and extensive washing of the embryos, a dual method of preparing RNAs from the pellet of these blastoderm cells is performed. The cell pellet is frozen and the RNAs are first extracted by a Trizol method (Tri Reagent (Invitrogen cat 15596-018) in an amount of 5 ml for around 1×10⁶ cells. After homogenization with the pipette, the mixture is centrifuged for 10 minutes at 400 g at 4° C., then 200 μl of chloroform per 1 ml of solution are added. The entirety is then emulsified, decanted at room temperature and the operation is repeated several times. The mixture is then centrifuged for 15 minutes at 400 g at 4° C. and the aqueous phase is transferred to a new tube. 500 μl of isopropanol per ml of mixture is added. The entirety is then precipitated by centrifugation for 15 minutes at 400 g at 4° C., the pellet is washed in ethanol at 70° C., dried and placed in 200 to 400 μl of lysis buffer of the Quiagen RNA extraction kit. This second step of preparation/purification of the RNA is then followed according to the manufacturers instructions. This dual extraction provides better reproducibility results in the QRT-PCR analysis of the gene expression level.

The conditions for QRT-PCR detection are identical to those described above (cf. example 4).

The relative expression level of the genes in the stage X blastoderm cells indicates that certain genes are also very highly expressed in these cells by comparison with embryonic fibroblasts.

### Example 7

### Identification of the Genes involved in the Physiology of Avian Germ Cells (CGSC)

To identify the genes involved in the emergence and maintenance of chicken germ cells, the screening strategies used for the CESC cells were also applied to chicken embryonic gonad cells.

As in the approach used to study the genes of CESC cells, the gene expression level was analyzed in avian germ cells. The list of genes expressed in these cells makes it possible to identify genes very highly expressed in these cells (relative overexpression ratio greater than 100 and genes with a relative expression level between 100 and 10). Many of the genes have a more modest expression with a ratio between 2 and 10.

### Example 8

### Analysis of the Comparative Expression of Genes Between the Various Cell Types

By comparing the relative expression levels between the CESC cells, the CEGC cells and the stage IX-XII blastoderm cells, it is possible to obtain various lists of genes that appear to be genes marking characteristics of the different cell types (the raw results are provided in an appendix brut).
With stage IX-XII blastoderm cells

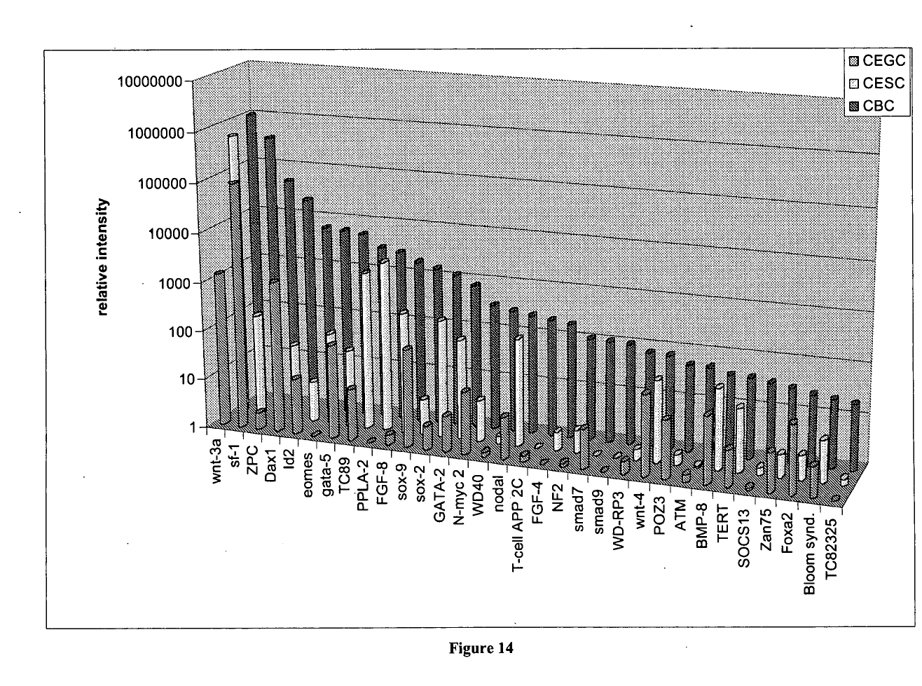

By combining the previous results of the various expression profiles, an expression combinatory specific to each cell type can be defined. Thus, when the expression levels are compared between the stage X blastoderm cells with the CESC cells in vitro, the stage X blastoderm cells overexpress (FIG. 14):
growth factors such as fgf-8, tgf-b2, fgf-4, wnt-14,
signaling players, such as smad-9, cis-1, smad-7, id2, smad-4,
transcription factors such as sf-1, gata-5, sox-9, sox-2,
structural genes such as WD40, WDRP-3, transporter abcg2,
regulator genes such as N-myc2, poz-3, atm,
other less characterized genes, such as T-cell phosphatase APP2C, ssb-1, nf-2,
the zpc gene, which appears to be very specific at this development stage.

This same comparison between the stage X blastoderm cells and the CEGC embryonic gonad cells indicate an overexpression (FIG. 14):
growth factors such as fgf-8, fgf-4, wnt-3a, wnt-11, tgf-b2,
signaling players, such as id2, smad-9, socs-1, cis-1, smad-7,
transcription factors such as eomes, sox-3, sox-2, 1P06, gata-2, gata-5, pax-6, fbx-15b, sox-9,
structural genes such as WD40, claudine-1, astacin, WDRP-3, abcg2,
regulator genes such as atm, N-myc-2, poz-2, poz-3,
other less characterized genes, such as ens-1, T-cell phosphatase APP2C, TC893, nf-2, ssb-1, TC823,
the zpc gene, which appears to be very specific at this development stage.

Thus, the combinatory best allowing the definition of stage X includes the genes most often expressed between the two situations:
fgf-4, tgf-b2,
id-2, smad-7, smad-9, snoN, SOCS13,
Cxcr-4,
gata-5, N-Cor1,
atm, bloom syndrome, enx-1, rad54b, rbm6, ssb-1,
nf-2, N-myc2, pml, T-cell APP2C, WDRP-3,
cpe1738, cwf16, TC82325, TC87, zan75,
Zpc.

For the stage X blastoderm cells, it is thus possible to note the presence of numerous signaling players such as factors fgf-4 and fgf-8, genes id-2, SOCS13, various members of the smad family, including snoN, acting to transmit the intracellular signals. Even though strong, the presence of these players is not exclusively specific.
The CESC cells

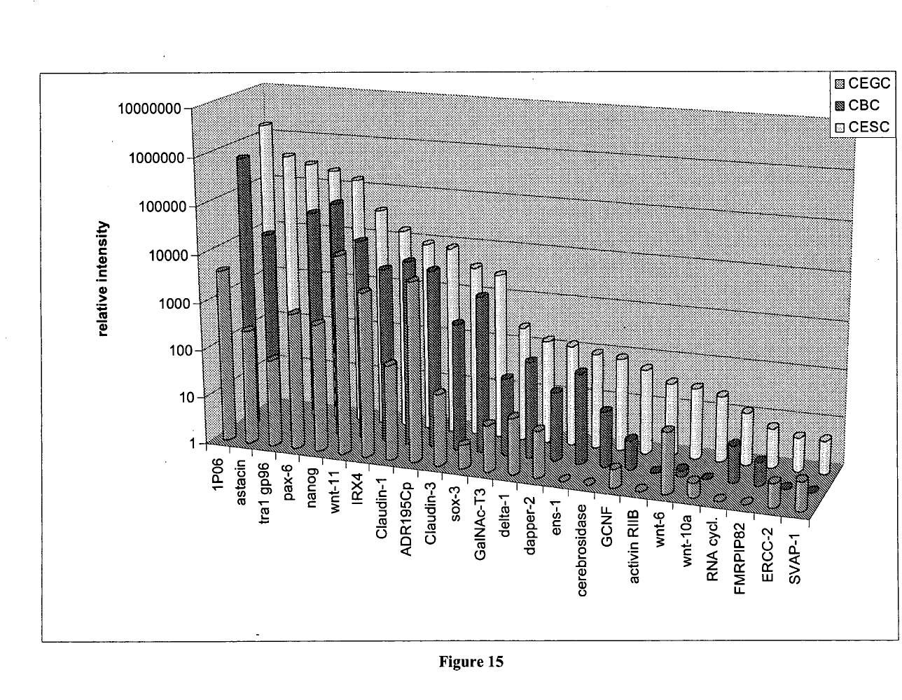

Identically, the CESC cells overexpress with respect to the stage X blastoderm cells (FIG. 15)
growth factors such as wnt-10, bmp-5, dapper-1,
signaling players, such as brap, TGF-RII,
transcription factors such as gcnf, TR-a, pax-6, 1P06, irx-4,
structural genes such as emerin, astacin, claudin-3, LAMP lectin, svap-1, dorfin,
marker genes such as tra-1, ch-tog, musashi,
other less characterized genes, such as FTT synthase, CG182, maturase K, RNA cyclase, FMRPI182.

And with respect to the CEGC embryonic gonad cells (FIG. 15):
growth factors such as wnt-3a, wnt-10, fgf-8, bmp-8, wnt-11, fgf-4, dapper-2,
signaling players, such as activin RII, brap,
transcription factors such as nanog, eomes, sox-3, 1P06, pax-6, gcnf, sox-2, gata-2, fbx-15b, irx-4,
structural genes such as astacin, claudin-3, claudin-1,
marker genes such as tra-1, ens-1,
other less characterized genes, such as TC896, maturase K, RNA cyclase, FMRPI182.

Thus, the combinatory best allowing the definition of the proliferating CESC cells includes the genes:
wnt-10a, wnt-11, dapper-1,
1P06, pax-6, gcnf, irx-4, sox-3,
astacin, claudin-3,
tra-1,
1P08-A09, slc38a, FMRPI182.

For the proliferating CESC cells, it is possible to mention the high proportion of transcription factors in the characteristic list of these cells. In particular, the genes nanog, 1P06 (oct-3/4—cf. below) and gcnf were isolated, cloned and studied in chickens for the first time during this study.

New genes were also identified, such as maturase, the gene of unknown function FMRPI182, of which the expression is associated with proliferation, the gene slc38-2 (undoubtedly a member of the solute carrier family), 1P08-A09, etc.
The CEGC cells

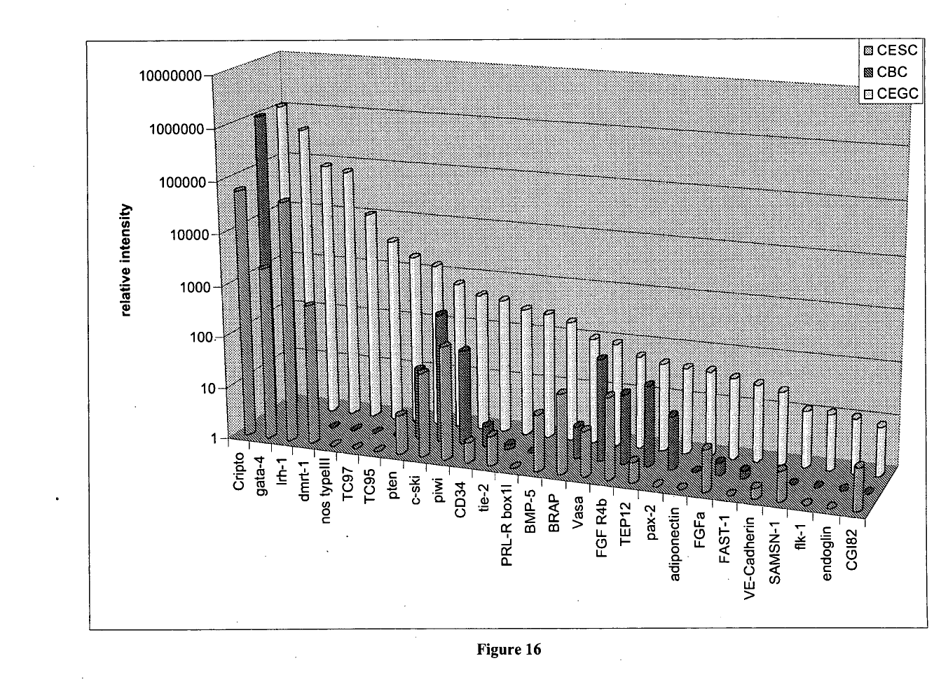

The embryonic gonad cells, subject to use of the entire tissue (cf. above), overexpress with respect to stage X blastoderm cells, the genes (FIG. 16):
growth factors such as bmp-5, dkk-3,
signaling players such as the endoglin receptor, TGF-RII, FGF-R, pten, wisp-1, smad-3, FGF-R, smarcd-3,
transcription factors such as pax-2,
structural genes such as emerin, VE-cadherine-2, HSPb7,
marker genes such as tie-2, piwi, musashi,
other less characterized genes such as CGI182, samsn-1, flj00188.

And with respect to the proliferating CESC cells, the genes (FIG. 16):
growth factors such as wnt-14, bmp-5, dkk-3,
signaling players such as the endoglin receptor, PRL-Rbox1, wisp-1, pten, cis-1, smad-7, smad-9, smad-4, TGF-RII, c-ski, smarcd-3,
transcription factors such as lhx-9, sf-1, gata-4, pax-2, dax-1, sox-9,
structural genes such as abcg-2, VE-cadherine-2,
marker genes such as tie-2, dmrt-1, piwi,
other less characterized genes such as TC95, KHKoc-1, flj00188, tep12, samsn-1,
germ cell marker genes such as vasa, piwi.

Thus, the combinatory making it possible to best defineallowing the definition of the CEGC embryonic gonad cells includes the genes:
BMP-2IK, BMP-5, dkk3,
Endoglin, FGF R, TGF RII, Wisp-1,
Gata-4, lhx-9,
Rab40B, smad3, Smarcd3,
TC95408, TC97694, FLJ00188,
tie2, tob.

These combinatorials are merely indicative and few genes appear to be very specific of a single cell type.

Certain genes identified have homologs in other species, of which the expression profiles are restricted to reproductive tissues such as deadend, vasa, wisp-1, tep12, and so on. By comparing the common expression profiles between stage X blastoderm cells and CESC cells (two cell types with germinal competence), we can identify a combinatorial of genes of which the expression is low or almost absent in CESC cells. This loss or significant decrease in markers of germ cells could be a consequence of the culture of stage X blastoderm cells. But no indication is possible with regard to the expression of these genes for a condition necessary and/or sufficient for germinal competence. The genes are:
wnt-14,
Brinp, cis-1, c-ski, prl-Rbox11, pten, sef, smad-4, smad-5,
Abcg2, clock, dmrt-1, lfng, morf,
Dax-1, pax-2, sf-1, sox-9,
Deadend, poz-3, slug, Ve-cadherinecadhérine-2,
Emx-2, fragilis4, TC136, tep12, tsc-22,
Tudor, vasa.

Similarly, even with variations in their expression level, some genes are common to the CESC and CEGC cells with the relative exclusion of stage X blastoderm cells; These genes:
cripto, BRAP, TGF RIII,
CGI82, ch-tog, ddx-25, Dorfin, emerin,
ERCC-2, KHKoc1, LAMP lectin,
Maturase, musashi, piwi, SAMSN-1, SVAP-1, TC86990,
TR-alpha.

The same analysis is also performed for genes expressed commonly between CESC cells and stage X blastoderm cells with the relative exclusion of CEGC cells. These genes are:
nodal, wnt-3a, wnt-11
Ras like
drg-1, ens-1, ews-1
Eomes, Fbx15b, gata-2, jsd3, pax-6, sox-2, sox-3
Ddx-28, Mago, nestin, POZ2
Arg NmethylASE, PP2447, RNA cyclase
A14TS, CES-c32, TC893, Translin
Claudin-1, syntaxin 16, WD40, WDFYP-2.

An analysis of the various genes that appear to be overexpressed in one or the other of the situations makes it possible to propose various models for maintaining the pluripotency in vitro, the control of the germinal competency in vivo, as well as the impact of culture conditions on the expression level of specific genes.

### Cytoskeleton

Concerning genes of the cytoskeleton, which ensure maintenance of the morphogenesis of CESC cells, only a limited number of genes have a significant beneficial expression differential. In particular, a gene having a WD motif, the gene WDRP-2 (TC190198) of which the motif involved in the protein-protein interactions at the level of the cytoskeleton is expressed more in the CESC cells than in the CEGC cells. The gene WD40 (TC189537) appears to be fairly specific to stage IX-XII blastoderm cells. It is noted that the beta-catenin gene is highly expressed in the two systems, suggesting that its regulation is more likely performed at the protein level. Conversely, the Claudin-1 gene (TC189974) involved in the “gap junctions” and the maintenance of strong cell-cell associations is highly expressed in CESC cells. This observations supports the electron microscopy analysis of the CESC cells in which the intra-cellular junctions are particularly large and well structured. We also detect astacin (TC 221952), a metalloprotease enzyme involved in the control of the extracellular matrix of cells enabling their movement and potential interactions with their environment.

### Growth Factors

The CESC cells and the GESC cells express, at variable levels, different members of the major families of growth factors and cytokines. Thus, numerous growth factors of the TGF/BMP families, the FGF family, the tyrosine kinase receptor factor family, the cytokine family of which the signal is transmitted by the gp130 pathway and the wnt family are expressed specifically in either the CESC cells or in the GESC cells.

Among the various players tested, the CESC cells thus express the nodal, FGF-8, wnt-3a, wnt-10a, BMP-8 and FGF-4 factors much more strongly than the CEGC cells. These last ones are in particular characterized by an overexpression of the wnt-14, TGF-b2 and BMP-5 factors.

It is noted that the nodal expression was involved in a complex pluripotency regulation system as mentioned for murine and human ES cells (Besser, 2004, Vallier et al., 2004).

The cripto gene, found both in the GESC cells and the CESC cells, is mentioned as an essential element in a “signature” of human ES cells (Bhattacharya et al., 2004).

In the mouse, the expression of FGF-8 was involved in the proliferation of PGCs, when they arrive after migration in the gonads (Kawase et al., 2004). The high expression of FGF-8 in the CESC cells may be a good indicator of a PGC “nature” of these cells, but the much higher level than in the GESC cells could be a sign of a disturbance in the activation pathway of this factor. Similarly, FGF-4 appears to be expressed around five times more in the CESC cells than in the GESC cells. It is noted that FGF-4 has been identified in the mouse as a privileged target of the transcription factor Oct-3/4 (Dailey et al., 1994; Ambrosetti et al., 2000) and is an essential player in the MESC cells both in vitro and in vivo (Yuan et al., 1995, Feldman et al., 1995, Wilder et al., 1997).

The situation of the two BMP factors is also particular, especially due to the strong involvement of the BMP-4 and BMP-8 factors in the control of the pluripotency of the MESC cells (Ying et al., 2001) and in the emergence and maintenance of murine germ cells in vitro (Hubner et al., 2003; Geijsen et al., 2004) and in vivo (Saitou et al., 2002). The destruction of the expression of BMP-4 by a loss of unction in the mouse results in early death of the embryo (8-9 days of development) by the absence of formation of mesodermal derivatives (Winnier et al., 1995). In these embryos, no PGC is detected (Lawson et al., 1999). This absence cannot be compensated by BMP-8. BMP-4 is itself necessary for early induction from the epiblast. The destruction of the expression of the BMP-8b gene results in a decrease in the proliferation of germ cells and a decrease in the engagement of the cells in meiosis (Zhao et al., 1996, Zhao et al., 1998).

For the sdf-1 gene, the expression of the messenger of this factor is correlated with that of its receptor, CXCR4, also detected specifically in the gonads, even if the positive cells in the entire tissue are not identified. The CESC cells do not have this property of expressing the factor or the receptor ensuring the guidance of germ cells because these two players are heavily involved in the control of germ cell migration, as shown both in the zebra fish (Doitsidou et al., 2002, Knaut et al., 2003), the mouse (Ara et al., 2003) and more recently in the chicken (Stebler et al., 2004). It would thus be possible to modify the tropism of the CESC cells by expressing, for example, the CXCR4 receptor in the CESC cells in order to obtain a more effective gonadic colonization. The high expression of CXCR-4 in the stage IX-XII blastoderm cells is an important element in the control of the migration of cells in the early embryo.

Another important player is the deadend gene, involved in the germ colonization process (Weidinger et al., 2003) and of which the ter mutation compromises the development of germ cells (Youngren et al., 2005). This gene is expressed more in the gonad cells than in the CESC cells.

The expression levels of the other factors and receptors tested are not as specific to the CESC cells or the GESC cells.

### Membrane Receptors

The TGFb/BMP and FGF receptor messengers are detected in all of the cell types tested, including the CEFs. Even if the physiological importance of these variations is unknown, it does not appear on a first approach that a real signaling deficit can be observed at the level of these receptors, on the condition that the level of the RNA detected is a good indicator of the protein level. Nevertheless, the activin IIB (U31223) receptor strongly expressed in CESC cells is detected, undoubtedly in relation to the high expression of nodal and cripto in these cells.

The CEGC cells express the receptors FGF-R (U48395), TGF-RII (AF202991) and TGF-RIII (L01121) in high proportions by comparison with the CESC cells. The expression profiles of the various genes of the tyrosine kinase families do not appear to be highly variable, even if the number of genes analyzed is not exhaustive.

Notably, the expression levels of c-kit, a SCF receptor, is very low in the CESC cells, contrasted with the expression observed in embryonic gonad cells. However, the importance of the SCF in maintaining the germ line is also demonstrated. This c-kit level would thus be a good indicator of the germinal nature of the cells, even if the c-kit receptor is expressed in many other tissues (hematopoiesis, nerve cells, etc.).

At the level of the LIF signaling pathway, we find expressions of the LIF-R receptor and the gp130 protein, which are also detected in the fibroblasts (Duong et al., 2002; Geissen et al., 1998). The avian LIF factor, recently cloned (Horiuchi et al., 2004) is detected only in the fibroblasts, which is consistent with the production of this cytokine in the other species and which suggests that the CESC cells do not appear to have autocrine production under the culture conditions used.

### Signaling

At the level of the signaling pathways, most of the players in the activation pathways of tyrosine kinase receptors, TGFb/BMP receptors and the gp130 jak/STAT pathway are generally expressed ubiquitously without significant variation between the various cell types. Nevertheless, some observations should be made. The PTEN gene (BM486819) is found to be highly expressed in CEGC cells as well as genes of the POZ domain, such as POZ3 (TC216137). Genes such as CIS-1 (TC216000), SSB-1 (TC210358), SOCS13 (TC217588), and so on are involved in the signalings by the various receptors as well as numerous genes involved in the GTPase pathway. The genes Smad-3 (TC187835), Smad-6 and smad-7 (TC194136), known for their regulatory action on TGF/BMP are also expressed more in the CEGC cells than in the CESC cells, even if we find a high expression in the CEFs. The genes Smad-1 (Tremblay et al., 2001) and Smad-5 (TC209770) (Chang et al., 2001), known to be heavily involved in the determinism of germ cells in the mouse are respectively more expressed in CESC cells and in CEGC cells. An expression in fibroblasts is also detected. The Smad-4 gene (TC207213) is preferentially expressed in CEGCs and is not, or is only slightly, detectable in the CESCs and in the CEFs. This Smad-4 gene appears in many situations as a key gene in the signaling of members of the TGFb/BMP family, because it is essential for the transcriptional control of genes dependent on these factors. Among the numerous defects associated with the inactivation of the smad-4 gene in the mouse, we see an absence of PGC formation (Chu et al., 2004) very similar to that observed in the mutants BMP-4 (Lawson et al., 1999) and BMP-8b (Ying et al., 2000; Loebel et al., 2003). The genes c-ski (TC219247) and SNoN (TC215069) also have specific expression differentials and are involved in these controls for proliferation and differentiation of these cells in the germ pathway. The expression level of these various players in the transmission of the signal of the TGF/BMP family can represent precious indicators of the “stem cell” or “germ cell” type of the cells studied. Any variation in the expression levels may be used to better detect the changes induced by the various manipulations of the candidate genes. These various genes themselves, in particular Smad-4, are very important candidates in the control of the germinal potential of a CES cell. It will therefore be particularly wise to test the effect or the destruction of these various genes both in vitro and in vivo.

The ras-like gene (TC201177) also has an expression more specific in the CESC cells than in the CEGC cells, as well as in stage IX-XII blastoderm cells. The murine ortholog gene was recently identified as a major player in the proliferation of MESC cells (Takahashi et al., 2003) and might be responsible for the oncogenic character of certain cell lines by the PI3 kinase pathway. However, the mutant MESC cells for this gene are able to colonize the germ line and no consequence on the descendency has been observed until now. The expression level in germ cells has not been documented, and an inhibition of expression in MESC cells has a negative effect on the proliferation rate. A hypothesis might be that the necessary decrease in the proliferation rate (via inhibition of this gene) would be a prerequisite for the differentiation of PGCs.

The other genes tested do not appear to have a major imbalance in their expression level. The regulation of the activity of other signaling players such as the proteins Stat, MAP kinase, erk, and so on is undoubtedly performed more at the protein level, in particular by the activation mechanisms associated with phosphorylations/dephosphorylations. This has been clearly demonstrated for the STAT-3 protein in the MESC cells.

At the level of nuclear protein factors, the situation appears to be complex, with regard to the large number of candidate genes. For the transcription factors, diagrammatically, various gene families appear to be particularly involved, such as that of the hormone nuclear receptors (HNR), and those of the sox genes and the gata genes.

The involvement of certain genes is directly associated with the epigenetic control of gene expression. The knowledge of epigenetic mechanisms during the development of the chicken or in the avian model is limited to pioneering discoveries of the expression mechanisms of globin genes, including the identification of isolating sequences (Felsenfeld, 1993) and to studies on the transcriptional control of the lysozyme gene (Kontaraki et al., 2000). By analogy with the other mammal or lower eukaryotic systems, various chicken genes involved in the epigenetic control of gene expression have been identified. Among those for which a high expression differential is observed, we find the gene dmrt-1 more highly expressed in embryonic gonads and genes DCSM, enx-1 more highly expressed in blastoderm cells. The expression of the dmrt-1 gene might be specific to Sertoli cells as in the murine model (Lei et al., 2004), but the detection of this gene in the germ cell fractions changes the discussion. Other genes have interesting expression profiles but are also expressed in fibroblasts.

### Telomerase Genes

Stem cells and embryonic stem cells have an intrinsic telomerase activity, in particular, the CESC cells (Pain et al., 1996). The chicken telomerase gene has just been cloned (Delany et al., 2004; Swanberg et al., 2004) and the expression level in the CESC cells appears to be high.

### Genes of the Sox Family

For the sox genes, the expression of the sox-9 gene (U12533) is more specific to CEGC cells, which appears to be consistent with the importance of the role of this gene in testicular formation of the mouse (Chaboissier et al., 2004, Vidal et al., 2001), but might be expressed preferentially in the support cells. The conservation of the role of this gene between mammals and birds is suggested (Morais da Silva et al., 1996) and this gene would be a direct target of the nuclear receptor sf-1 (Sekido et al., 2004) (NM 205077), a player also found to be highly expressed in CEGC cells in the presence of Dax-1 (AF202991).

Among the genes most specific to the CESC cells, we will mention the two genes sox-2 (U12532) and sox-3 (U12467). Strong associations have been demonstrated in the proteins SOX-2 and OCT-3/4 in the mouse at the level of certain promoters (Miyagi et al., 2004; Nishimoto et al., 1999), in particular at the level of the promoter of FGF-4 (Dailey et al., 1994; Ambrosetti et al., 2000). The 1P06 gene, an avian homolog of the mammal Oct-3/4 gene (cf. below, example 9) is very highly expressed in CESC cells and appears to be one of the best markers of these cells.

### Genes of the Gata Family

The gata genes also show particular expression profiles. The gata-4 gene (U11887) is expressed preferentially in the embryonic gonad cells, which expression can be related to the presence of Sertoli cells (Imai et al., 2004, Lavoie et al., 2004). The genes gata-2 (X56930) and gata-5 (U11888) are more highly expressed in CESC cells, in particular with a high overexpression of the gata-5 gene in the blastoderm cells. The messenger of the gat-2 gene was detected in germ cells (Siggers et al., 2002) and at the level of the chicken early embryo (Sheng et al., 1999). The pleiotropic action of the gata genes is modulated and controlled by the association with various partners at the level of close and/or common response elements in the promoters of the target genes. Thus, if one of the preferred partners in the hematopoietic system is one of the genes of the ets family, the combinations are different in other proliferation and differentiation systems. Thus, gata gene response elements are identified with BMP response elements (BRE elements) in the promoter of the smad-7 gene (Benchabane et al., 2004). This smad-7 gene is also more highly expressed in embryonic gonad cells than in the CESC cells.

The genes of the Id family appear to be undeniable players in the control of the BMP action. The Id2 gene appears to be more highly expressed in embryonic gonad cells, and in particular in the blastoderm cells, than in the CESC cells. This gene could be at the center of the regulation of the proliferation/differentiation of the CESC cells, in an approach similar to the murine model (Ying et al., 2003, Kowanetz et al., 2004).

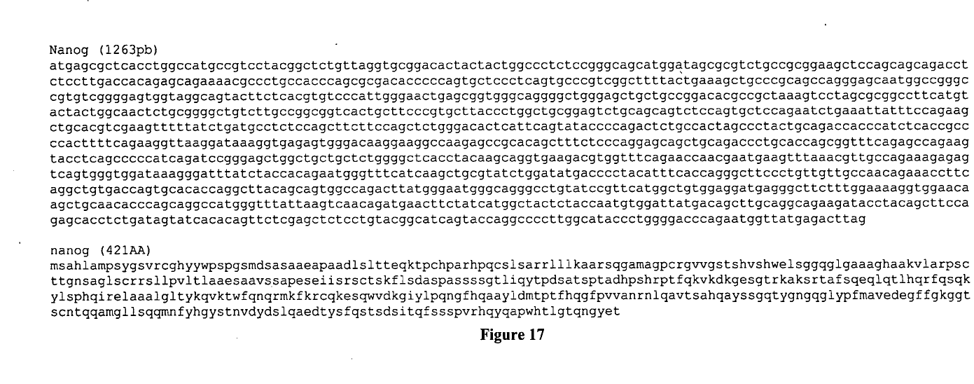

In these various processes described in the literature, we often find the partner YY1 (Kurisaki et al., 2003) (also found to be highly expressed in CESC cells, but of which the expression level is relatively ubiquitous) and a player of the NKx family, as in the case of cardiac differentiation (Lee et al., 2004). However, the nanog gene, currently identified exclusively in mammals, belongs to this Nkx gene family (Chambers et al., 2003). In the search for homology at the level of the published chicken genome sequence, the chicken nanog gene was identified (FIG. 17). The function of this player is still largely unknown in the mouse system. The ENS-1 protein, identified in the gene trapping approach (Acloque et al., 2004), also has an expression profile that is particularly beneficial, with a very high overexpression in the CESC cells and rapid disappearance of its messenger when the cells are caused to differentiate (Acloque et al., 2001).

Even though the homolog of the utf-1 gene known to be a target of the complex Oct-3/4/sox-2 (Nishimoto et al., 1999) has not yet been identified in the chicken, we will mention that in transfection experiments, the promoters of the mouse genes oct-3/4, sox-2 and utf-1 are specifically activated in the CESC cells, suggesting that the mechanisms for regulating the expression of these various actors are preserved and functional in the avian CESC cells (cf. example 12). This transactivation is not detected in a chicken embryonic fibroblast.

### Nuclear Hormone Receptors

For hormone nuclear receptors, the dax-1 gene appears to be very highly overexpressed in blastoderm cells. This gene is known to have a negative (Swain et al., 1998) but essential role (Meeks et al., 2003) in germ differentiation, in particular by its antagonist action on that of the sf-1 gene (Crawford et al., 1998) This dax-1 gene might itself be positively controlled by sf-1 and negatively controlled by COUP-TF (Yu et al., 1998). The expression of dax-1 would also be dependent in certain systems on the activation by wnt-4 (Mizusaki et al., 2003). However, wnt-4 is one of the factors found to be more highly expressed in gonads than in the CESC cells. The genes coding for the proteins associated with HNR are also represented and have particular differentials, such as the N-Corl gene (TC201157).

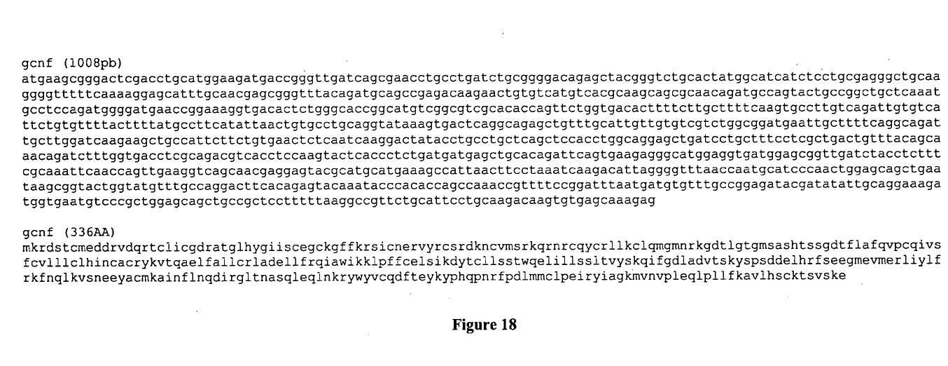

The gcnf gene of which we have determined the entire sequence (FIG. 18) is found to be very highly and specifically expressed in CESC cells and its antagonist sf-1 action at the level of the promoter of the Oct-3/4 gene is demonstrated in the mouse (Furhmann et al., 2001).

The sf-1 gene is found to be almost exclusively expressed in embryonic gonad cells. However, sf-1 and lrh are two homologs of FTZ-Fi, of which the role in early development has been documented (Kudo et al., 1997; Fayard et al., 2004). An additional regulation level and a possible link between the actions of the hormone nuclear receptors and the other partners (such as sox-9, for example) could be achieved via sumoylation. The importance of this post-translational modification in the control of the transcription and the integrity of the genome is constantly growing (Muller et al., 2004; Seeler et al., 2003). Sumoylation modifies both the cell distribution and the interactions between factors (Chen et al., 2004; Komatsu et al., 2004). Other receptors known to be involved in the determinism of mouse germ cells such as ERR (Mitsunaga et al., 2004) do not appear, in the system studied, to have high variations in their expression level.

The receptors RAR, RXR, TR, are more specifically involved in cell differentiation. Their expression kinetics and the variations in their expression level have been analyzed in example 5.

### RNA Binding Proteins

Among the proteins involved in the control of gene transcription in germ cells, we find proteins that bind the RNAs, i.e. “RNA binding proteins”. These proteins have been identified in particular in the fruit fly and are involved in the control of embryo polarity (Huynh et al., 2004a) and the determinism of germ cells. In another model species, C. elegans, the hypothesis of the pre-formation of germ cells is also and schematically based on the existence and the role of these proteins. However, in these two models, the control of the activation by phosphorylation of the RNA polymerase II, which activation is necessary for transcription, is not performed by these “RNA binding proteins” (Seydoux et la., 1997). In mammals, it has been shown that some of these genes are also essential to maintaining the germ line in the mouse (Wang et al., 2004; Tsuda et al., 2003; Tanaka et al., 2000). However, the origin of murine germ cells appears to be more related to a phenomenon of induction by various players related to the three-dimensional cell environment and to the action of factors such as BMP-4 and BMP-8. All of these inductions in the end control the expression levels of the various major players such as Oct-3/4 (Saitou et al., 2003). This inductive origin appears to be reinforced by obtaining germ cells in vitro from the differentiation of mouse MESC cells (Hubner et al., 2003, Geijsen et al., 2004, Toyooka et al., 2003).

In the chicken, the theory of pre-formation was revivified by the identification of vasa positive cells from the early stages of the chicken embryo (Extavour et al., 2003; Tsukenawa et al., 2000). In this hypothesis, the role of certain “RNA binding proteins” may be essential.

Of the sixty or so genes of this family of which the expression level was tested, we detected the genes bruno (AB050497), tudor (TC213378), nanos (TC223629), vasa (AB004836) as being much more highly expressed in CEGC cells than in the CESC cells, but also in the blastoderm cells, undoubtedly corresponding to the presence of competent cells at the germ level in these cells. The CESC cells are positives for the vasa gene, but have a very low contribution to the germ line once they are kept in culture. Also, the relative expression level appears to be an essential element in this germinal competence. Some, such as the nanos gene, have an almost exclusive expression specificity for the CEGC cells. The mago gene has a higher expression in the blastoderm cells by comparison with the two CESC and CEGC cell types. We will also mention the genes characterized by a “Dead box”, a characteristic motif of RNA helicase proteins able to bind RNAs (Rocak et al., 2004). The ddx 28 gene (TC211748) is well expressed in CESC cells as well as the homolog of the cyclase RNA (TC214888). New homolog molecules of fruit fly and/or C. elegans proteins have yet to be identified, in particular the homologs of oskar, pumilio, aubergine, maelstrom and so on. In mammals, the role of these “RNA binding” proteins in controlling the nature of germ cells is unknown, but it appears, according to the results obtained in the fruit fly, that any disruption in the activity of these genes causes a loss of the germ line. The proteins appear to act on the control of the cytoplasm/core localization of the messengers that they recognize and therefore on the translation efficacy of these target messengers (Huynh et al., 2004; Yano et al., 2004; Hachet et al., 2004, Palacios et al., 2004). The mechanisms of action of these proteins and their RNA are constantly increasing in complexity, in particular by a possible regulation of the expression level of these messengers by the RNAi and the RISC complex (“RNA-induced Silencing complex”) (Tomari et al., 2004).

The piwi gene, involved in the asymmetric divisions and in maintaining the germ line of numerous species (Cox et al., 1998; Kuromochi et al., 2004) is more highly expressed in CEGC cells than in the CESC cells. The blastoderm cells appear to be less expressed in piwi than the CESC and CEGS cells. This piwi gene belongs to the family of Argonaute proteins and is one of the essential components of the RISC complex. It contains a PAZ domain that may link the RNAs and act as an endonuclease at the level of the mRNA in the siRNA/mRNA degradation duplex (Song et al., 2004; Tahbaz et al., 2004). In addition, the expression level of the dicer gene, which ensures the production of the siRNA and which has also been identified in the chicken (Fukagawa et al., 2004) appears to be equivalent in the CESC and CEGC cells.

### Other Genes

Among the other genes that have high expression differentials according to the cell types compared, we can note the presence of tra-1 (NM 204289), specific to CESC cells and blastoderm cells, the ens-1 gene, described above (Acloque et al; 2001), as a CESC cell marker, the fbx15 and fbx15b genes (Tokuzawa et al; 2003), T-box genes involved in the physiology of the MESC cells, but of which the role remains unknown, the IRX4 iroquois homeobox gene, of the family of Nkx transcription factors (Houweling et al., 2001). Numerous other genes have an expression differential.

### Unknown Genes

Genes of which the function is at present completely unknown have an expression differential between the various cell types tested. We will thus mention the genes TC976 (TC227369), TC954 (TC203410), FLJ00188 (TC 196697) overexpressed in CEGC cells, the genes TC869 (TC192927), 1P08-A09 (TC189639), TC823 (TC209084), slc38a2 (TC187360), TC896 (TC196399), FPPsynthase (TC211235), TC874 (TC193590) and CES-c32 (TC83694) overexpressed in CESC cells. We will mention the genes CPE1738 (TC214741) more highly expressed in blastoderm cells. The TC976 gene has a partial homology with the mouse ASB-6 protein containing an ankyrin motif and a SOCS (Suppressor of Cytokine Signaling) box (Wilcox et al., 2004, Kim et al., 2004). These ASB genes are often expressed in germ cells and the ASB14 gene is found to be expressed preferentially in the CESC cells and the CEGC cells, but little in blastoderm cells. One of the hypotheses of the function of these genes is that the SOCS domain would be involved in the specific germinal compartmentalization of certain messengers and would promote their degradation in the soma (DeRenzo et al., 2003). This mechanism would exist in parallel and can be complementary to that performed by the “RNA binding proteins”.

### References

Acloque et al., (2001) Mech Dev. 103: 79-91.
Acloque et al., (2004) Nucleic Acids Res. 32: 2259-71.
Aida M et al. (2004) Cell 119: 109-20.
Allen J D et al. (2002) C. Mol Cancer Ther. 1: 417-425.
Ambrosetti D C et al. (2000) J Biol Chem. 275: 3387-97.
Anderson R et al. (2000) Mech Dev. 91: 61-8.
Ara T et al. (2003) Proc Natl Acad Sci USA. 100: 5319-23.
Arai F et al. (2004) Cell 118: 149-61.
Barth J L et al. (1998) Exp Cell Res. 238: 430-8.
Benchabane H, Wrana J L. (2003) Mol Cell Biol. 23: 6646-61.
Besser D. (2004) J Biol Chem. 279: 45076-84.
Bhattacharya et al., (2004). Blood 103: 2956-64.
Biniszkiewicz D et al. (2002) Mol Cell Biol. 22: 2124-2135.
Blackwell T K. (2004) Curr Biol. Mar. 23, 2004; 14(6):R229-30.
Blanpain C et al. (2004) Cell 118: 635-48.
Boeuf H et al. (1997) J Cell Biol. 138: 1207-17.
Buaas F W et al. (2004) Nat Genet. 36: 647-52.
Cai J et al. (2004) Exp Hematol. 32: 585-598.
Carsience R S et al. (1993) Development 117: 669-75.
Cartwright P et al. (2005) Development 132: 885-96.
Castrillon D H er al. (2000) Proc Natl Acad Sci USA. 97: 9585-90.
Chaboissier M C et al. (2004) Development 131: 1891-901.
Chambers I et al. (2003) Cell 113: 643-55.
Chambers I, Smith A. (2004) Oncogene 23: 7150-60.
Chang H, Matzuk M M. (2001) Mech Dev. 104: 61-7.
Charge S B, Rudnicki M A. (2004) Physiol Rev. 84: 209-238.
Chen W Y et al. (2004) J Biol Chem. 279: 38730-5.
Chu G C et al. (2004) Development 131: 3501-12.
Cossu G, Bianco P. (2003) Curr Opin Genet Dev. 13: 537-42.
Costoya J A et al. (2004) Nat Genet. 36:653-9.
Coumoul X et al. (2004) Nucleic Acids Res. 32: 85.
Cox D N et al. (1998) Genes Dev. 12: 3715-27.
Crawford P A et al. (1998) Mol Cell Biol. 18: 2949-56.
Daheron L et al. (2004) Stem Cells. 22: 770-778.
Dailey L et al. (1994) Mol Cell Bio. 14: 7758-69.
Damiola F et al. (2004) Oncogene 23: 7628-43.
Dani C et al. (1998) Dev Biol. Nov. 1, 1998; 203(1): 149-62.
De Felici M et al. (1993) Dev Biol. 157: 277-80.
Delany M E, Daniels L M. (2004) Gene 339:61-9.
DeRenzo C et al. (2003) Nature 424: 685-9.
Doitsidou M et al. (2002) Cell 111: 647-59.
Duong C V et al. (2002) Development 129: 1387-96.
Evans M J, Kaufman M H. (1981) Nature 292: 154-6.
Extavour C G, Akam M. (2003) Development 130: 5869-84.
Eyal Giladi & Kovak (1976).
Fabioux C et al. (2004) Biochem Biophys Res Commun. 320: 592-8.
Fayard E et al. (2004) Trends Cell Biol. 14: 250-60.
Feldman B et al. (1995) Science 267: 246-9.
Felsenfeld G. (1993) Gene. 135: 119-24.
Fuhrmann G et al. (2001) Dev Cell. 1: 377-87.
Fujiwara Y et al. (1994) Proc Natl Acad Sci USA. 91: 12258-62.
Fukagawa T et al. (2004) Nat Cell Biol. 6: 784-91.
Furhmann G et al., (2001) Dev Cell. 1: 377-87.
Galli R et al. (2000) Nat Neurosci. 3: 986-991.
Gaudet F et al. (2004) Mol Cell Biol. 24: 1640-168.
Geijsen N et al. (2004) Nature 427: 148-54.
Geissen M et al. (1998) Development 125: 4791-801.
Ginis I et al. (2004) Dev Biol. 269: 360-380.
Goodell M A et al. (1997) Nat Med. 3: 1337-45.
Grabarek J B et al. (2003) Biotechniques. April 2003; 34(4): 734-6, 739-44.
Gu P et al., (2005) Mol Cell Biol. 25: 8507-19.
Ha J Y et al., (2002); Theriogenology.58: 1531-9.
Hachet O, Ephrussi A. (2004) Nature 428: 959-63.
Hamada H et al. (2005) Cancer Sci. 96: 149-156.
Hamburger& Hamilton (1951.)
Hattori N et al. (2004) Genome Res. 14: 1733-1740.
Hogan B et al. (1994). Isolation, Culture and Manipulation of Embryonic Stem cells. In Manipulating the Mouse Embryo. A laboratory Manual, Second Edition, pp 253-291. CSHL Press.
Hong Y et al. (1996) Mech Dev. 60: 33-44.
Hong Y et al. (2004) Mech Dev. 121: 933-43.
Horiuchi H et al. (2004) J Biol Chem. 279: 24514-20.
Houweling A C et al. (2001) Mech Dev. 107: 169-174.
Hubner K et al. (2003) Science 300: 1251-6.
Humphrey R K et al. (2004) Stem Cells 22: 522-30.
Huynh J R et al. (2004 Dev Cell. 6: 625-35.
Imai T et al. (2004) Mol Cell Endocrinol. 214: 107-15.
Ivanova N B et al. (2002) Science 298: 601-4.
Jiang Y et al. (2002) Exp Hematol. 30: 896-904.
Jiang Y et al. (2002) Nature 418: 41-4 9.
Karagenc L et al. (1996) Dev Genet. 19: 290-301.
Karagenc L, (2000). Poult Sci. 79: 80-5.
Katahira T, Nakamura H. (2003) Dev Growth Differ. 45: 361-7.
Kawase E et al. (2004) Mol Reprod Dev. 68: 5-16.
Kehler J et al. (2004) EMBO Rep. 5.
Kim K S et al. (2004) Zygote 12: 151-6.
Knaut H et al. (2002) Curr Biol. 12: 454-66.
Knaut H et al. (2003) Nature 421: 279-82.
Komatsu T et al. (2004) Mol Endocrinol. 18: 2451-62.
Komiya T et al. (1994) Dev Biol. 162: 354-63.
Kontaraki J et al. (2000) Genes Dev. 14: 2106-22.
Koshimizu U et al. (1995) Dev Biol. 168: 683-5.
Kowanetz M et al., (2004) Mol Cell Biol. 24: 4241-54.
Krovel A V, Olsen L C. (2004) Dev Biol. 271: 190-7.
Kudo T, Sutou S. (1997) Gene 197: 261-8.
Kunath T et al. (2003) Nat Biotechnol. May 2003; 21(5):559-61. Epub Apr. 7, 2003.
Kuramochi-Miyagawa S et al. (2004) Development 131: 839-49.
Kurisaki K et al. (2003) Mol Cell Biol. 23: 4494-510.
Kuromochi et al., 2004.
Lakshmipathy U, Verfaillie C. (2005) Blood Rev. 19: 29-38.
Lasko P F, Ashburner M. (1988) Nature 335: 611-7.
Lassalle B et al. (2004) Development 131: 479-487.
Lassalle B et al. (2004) Development 131: 479-87.
Lavoie H A et al. (2004) Mol Cell Endocrinol. 227: 31-40.
Lawson K A et al. (1999) Genes Dev. 13:424-36.
Lee K H et al. (2004) Development 131: 4709-23.
Lei N, Heckert L L. (2004) Mol Cell Biol. 24: 377-88.
Levavasseur F et al. (1998) Mech Dev. 74: 89-98.
Lickert H et al. (2005) Development. June 2005; 132(11):2599-609. Epub Apr. 27, 2005.
Lin T et al. (2005) Nat Cell Biol. 7:165-71.
Loebel D A et al., 2003 Dev Biol. 264:1-14. Review.
Martin G R. (1981) Proc Natl Acad Sci USA. 78: 7634-8.
Matsuda T et al. (1999) EMBO J. 18: 4261-9.
Mayhall E A et al. (2004) Curr Opin Cell Biol. 16: 713-720.
Meeks J J et al. (2003) Nat Genet. 34: 32-3.
Metzger D et al. (1995) Proc Natl Acad Sci USA. 92: 6991-5.
Minasi M G et al. (2002) Development 129: 2773-2283.
Mitsui K et al. (2003) Cell 113: 631-42.
Mitsunaga K et al. (2004) Mech Dev. 121: 237-46.
Miyagi S et al. (2004) Mol Cell Biol. 24: 4207-20.
Mizusaki H et al. (2003) Mol Endocrinol 17: 507-19.
Mochizuki K et al. (2001) Dev Genes Evol. 211: 299-308.
Morais da Silva S et al. (1996) Nat Genet. 14: 62-8.
Mukhopadhyay M et al. (2003) Development 130: 495-505.
Muller S et al. (2004) Oncogene 23: 1998-2008.
Nakamura H et al. (2004) Mech Dev. 121: 1137-43.
Nichols J et al. (1996) Mech Dev. July 1996; 57(2): 123-31.
Nichols J et al. (2001) Development 128(12): 2333-9.
Nishimoto M et al. (1999) Mol Cell Biol. 19: 5453-65.
Nishimoto M et al. (1999) Mol Cell Biol. 19: 5453-65.
Niwa H et al. (1998) Genes Dev. 12: 2048-60.
Niwa H et al. (2000) Nat Genet. 24: 372-6.
Niwa H et al. (2002) Mol Cell Biol. 22: 1526-36.
Ohinata Y, (2005). Nature 436: 207-13.
Okumura-Nakanishi S et al. (2005) J Biol Chem. 280: 5307-5317.
Pain B et al. (1996) Development 122: 2339-48.
Pain B et al., (1999) Cells Tissues Organs. 165: 212-9. Review.
Palacios I M et al. (2004) Nature 427: 753-7.
Park T S et al., (2003) Mol Reprod Dev. 65: 389-95.
Pekarik V et al. (2003) Nat Biotechnol. 21: 93-6. Erratum in: Nat Biotechnol. 21: 199.
Petitte J N et al. (1990) Development 108: 185-9.
Ramalho-Santos M et al. (2002) Science 298: 597-600.
Raz R et al. (1999) Proc Natl Acad Sci USA. 96: 2846-51.
Reik W et al. (2001) Science 293: 1089-1093.
Robertson E J. (1987). Embryo derived stem cells. In teratocarcinomas and Embryonic Stem Cells: a Practical Approach (Ed E.J. Robertson), pp 71-112. Oxford IRL Press.
Rocak S, Linder P. (2004) Nat Rev Mol Cell Biol. 5: 232-41.
Saitou M et al. (2002) Nature 418: 293-300.
Schisa J A et al. (2001) Development 128: 1287-98.
Seale P et al. (2004) Dev Biol. 275: 287-300.
Seeler J S, Dejean A. (2003) Nat Rev Mol Cell Biol. 4: 690-9.
Sekido R et al. (2004) Dev Biol. 274: 271-9.
Seydoux G, Dunn M A. (1997) Development 124: 2191-201.
Shamblott M J et al. (1998). Proc Natl Acad Sci USA. 95: 13726-31. Erratum in: Proc Natl Acad Sci USA 96: 1162.
Sheng G, Stern C D. (1999) Mech Dev. 87: 213-6.
Shibata N et al. (1999) Dev Biol. 206: 73-87.
Shiota K et al. (2002) Genes Cells 7: 961-969.
Shizuru J A et al. (2005) Annu Rev Med.; 56: 509-38.
Siggers P et al. (2002) Mech Dev. 111: 159-62.
Song J J et al. (2004) Science 305: 1434-7.
Song X et al. (2002) Science 296: 1855-7.
Song X et al. (2004) Development 131: 1353-64.
Soodeen-Karamath S. Gibbins A M. (2001) Mol Reprod Dev. 58: 137-48.
Stebler J et al. (2004) Dev Biol. 272: 351-61.
Sumi T et al. (2004) Stem Cells 22: 861-72.
Swain A et al. (1998) Nature 391: 761-7.
Swanberg S E et al. (2004) Dev Dyn. 231: 14-21.
Tada T et al., (1998) Dev Genes Evol.207: 551-61.
Tahbaz N et al. (2004) EMBO Rep.5: 189-94.
Takahashi K et al. (2003) Nature 423: 541-5.
Tanaka S S et al. (2000) Genes Dev. 14: 841-53.
Thomson J A et al. (1995) Proc Natl Acad Sci USA. 92: 7844-8.
Thomson J A et al. (1998) Science 282: 1145-7. Erratum in: Science 282: 1827.
Thoraval P et al. (1994) Poult Sci.73: 1897-905.
Thoraval P et al. (1994) Poult Sci.73: 1897-905.
Tokuzawa Y et al. (2003) Mol Cell Biol. 23: 2699-2708.
Tomari Y et al. (2004) Cell 116: 831-41.
Toyooka Y et al. (2003) Proc Natl Acad Sci USA. 100: 11457-62.
Tremblay K D et al. (2001) Development 128: 3609-21.
Tsuda M et al. (2003) Science 301: 1239-41.
Tsunekawa N et al. (2000) Development 127: 2741-50.
Vallier L et al. (2004) et al. Dev Biol. 275: 403-21.
Velculescu V E et al. (1995) Science 270: 484-7.
Vidal V P et al. (2001) Nat Genet. 28: 216-7.
Wagers A J, Weissman I L. (2004) Cell 116: 639-648.
Wang Z, Lin H. (2004) Science 303: 2016-9.
Weidinger G et al. (2003) Curr Biol. 13: 1429-34.
Wilcox A et al. (2004) J Biol Chem. 279: 38881-8.
Wilder P J et al. (1997) Dev Biol. 192: 614-29.
Winnier G et al. (1995) Genes Dev. 9: 2105-16.
Wurmser A E et al. (2004) Science 304: 1253-5.
Xie T, Spradling A C. (2000) Science 290: 328-30.
Yamashita Y M et al. (2003) Science 301: 1547-50.
Yano T et al. (2004) Dev Cell. 6: 637-48.
Yeom Y I et al. (1996) Development 122: 881-94.
Ying Q L et al. (2003) Cell 115: 281-92.
Ying Y et al. (2000) Mol Endocrinol. 14: 1053-63.
Ying Yet al., (20041) Proc Natl Acad Sci USA. 98: 7858-62.
Yoon C et al. (1997) Development 124: 3157-65.
Yoshida K et al. (1994) Mech Dev. February 1994; 45(2) :163-71.
Yoshimizu T et al. (1999) Dev Growth Differ. 41: 675-84.
Youngren K K et al., (2005) Nature 435: 360-4.
Yu R N et al. (1998) Mol Endocrinol. 12: 1010-22.
Yuan H et al. (1995) Genes Dev. 9: 2635-45.
Zhang et al. (2003) Nature 425: 836-41.
Zhao G Q et al. (1996) Genes Dev. 10: 1657-69.
Zhao G Q et al. (1998) Development 125; 1103-12
Zhou S et al. (2001) Nat Med. 7: 1028-34.

1. Combination of markers allowing the characterization of avian cells of StX-type, stem cells or germ cells, including at least two markers chosen either from the same group or from two different groups:
a. a marker of a target gene preferentially expressed in StX cells chosen from the following genes: 1P06, 2contig58, 60S-L14, ATM, Bloom syndrome, BTEB4, CD9, CHD helicase, Clock, cwf16 (FLJ10374), CXCR4, Dnmt2, enxl (ho-zeste 2), eomes, EWS, FGF-4, GATA-5, HOJ-1, N-AGN6P deacetylase, N-Cor1, NF2, p53, pml, rbm6, SA-2, SA-3, SARA, SCYE1, SEF, sf-1, SnoN, SOCS13, SSB-1, TC87479, T-cell APP 2C, TGF-beta2, WD40/FYVE-d protein 2, WD-RP3, Zan75, ZPC and combinations thereof, and/or
b. a marker of a target gene preferentially expressed in stem cells chosen from the following genes: 1P06, 1P08-A09, activin RIIB, astacin, Claudin-3, dapper-1, Dorfin, FPP synthase (fps), GalNAc-T3, gcnf, HSPb7, IRX4, LMX, pax-6, Slc38a2, sox-3, tra1 gp96, wnt-10a, wnt-11 and combinations thereof, and/or
c. a marker of a target gene preferentially expressed in germ cells chosen from the following genes: adiponectin, BMP-2IK, bruno like, CD34, CDK5 activator 1, dkk1, dkk3, DMRT1, emx2, endoglin, FAST-1, FGF R, FLJ00188, flk-1, gata-4, gcl, LHX9, NOS type III, plzf, PRL-R box1l, PTEN, SAMSN-1, slug, smad3, Smarcd3, sox-9, Strat8, TACC2, TC95408, TC97694, TGF RII, TGF RIII, tie2, tie-2, TR-alpha, VE-Cadherin, vera, Wisp-1 and combinations thereof.

2. Combination according to claim 1, characterized in that the marker of a target gene preferentially expressed in StX cells is chosen from the markers of the following genes: 1P06, ATM, CXCR4, eomes, FGF-4, GATA-5, NF2, SOCS13, SSB-1, TC87479, T-cell APP 2C, TGF-beta2, WD-RP3, ZPC and combinations thereof.

3. Combination according to one of claims 1 or 2, characterized in that it includes at least one marker of the ZPC gene.

4. Combination according to one of claims 1 to 3, characterized in that the marker of a gene preferentially expressed in stem cells is chosen from the markers of the following genes: 1P06, activin RIIB, astacin, Claudin-3, dapper-1, FPP synthase (fps), GalNAc-T3, gcnf, LMX, pax-6, tra1 gp96, wnt-10a and combinations thereof.

5. Combination according to one of claims 1 to 4, characterized in that it includes at least one marker of the 1P06 and tra-1 genes.

6. Combination according to one of claims 1 to 5, characterized in that the marker of a target gene preferentially expressed in germ cells is chosen from the markers of the following genes: adiponectin, DMRT1, endoglin, FAST-1, FGF R, FLJ00188, gata-4, LHX9, plzf, PRL-R boxll, PTEN, Strat8, TGF RIII, Wisp-1 and combinations thereof.

7. Combination according to one of claims 1 to 6, characterized in that it includes at least one marker of the dmrt-1 and vasa genes.

8. Combination according to one of claims 1 to 7, characterized in that the markers are chosen from the antibodies able to bind specifically to the expression products of a target gene, mRNA, cDNA or polypeptide, or a fragment of these, and the nucleic acid fragments able to hybridize specifically with the mRNA expressed by said genes or the corresponding cDNA, or fragments of these.

9. Combination according to one of claims 1 to 8, characterized in that it is assembled on a single support, preferably a standard support.

10. Combination according to claim 9, characterized in that it is in the form of a DNA matrix, including a support on which the nucleic acid fragments susceptible to hybridize with the target genes are arranged, preferentially in a standard way.

11. Combination according to claim 10, characterized in that it is arranged on a DNA chip.

12. DNA chip, characterized in that it includes a combination of markers according to one of claims 1 to 11.

13. Process for characterizing an avian cell including the analysis of the expression of genes expressed in said cell, using a combination of markers according to one of claims 1 to 11 and the characterization of the phenotype of the analyzed cells.

14. Process of growth of avian cells including the culture of cells in a suitable culture medium and the characterization of said cells by using a process according to claim 13.

15. Process according to claim 14, characterized in that some cells are isolated, these cells being selected from StX cells, stem cells and germ cells.

16. Process according to one of claims 14 or 15, characterized in that germ cells are obtained from StX cells, with the StX cells being cultivated in a suitable culture medium without the addition of an inactivated “feeder” layer.


SEQUENCE LISTING (376 sequences)

SEQ ID NO: 1 - DNA, 1263 bp, Gallus gallus
atgagcgctcacctggccatgccgtcctacggctctgttaggtgcggacactactactggccctctccgggcagcatggatagcgcgtctgccgcggaagctccagcagcagacctctccttgaccacagagcagaaaacgccctgccacccagcgcgacacccccagtgctccctcagtgcccgtcggcttttactgaaagctgcccgcagccagggagcaatggccgggccgtgtcggggagtggtaggcagtacttctcacgtgtcccattgggaactgagcggtgggcaggggctgggagctgctgccggacacgccgctaaagtcctagcgcggccttcatgtactactggcaactctgcggggctgtcttgccggcggtcactgcttcccgtgcttaccctggctgcggagtctgcagcagtctccagtgctccagaatctgaaattatttccagaagctgcacgtcgaagtttttatctgatgcctctccagcttcttccagctctgggacactcattcagtataccccagactctgccactagccctactgcagaccacccatctcaccgccccacttttcagaaggttaaggataaaggtgagagtgggacaaggaaggccaagagccgcacagctttctcccaggagcagctgcagaccctgcaccagcggtttcagagccagaagtacctcagcccccatcagatccgggagctggctgctgctctggggctcacctacaagcaggtgaagacgtggtttcagaaccaacgaatgaagtttaaacgttgccagaaagagagtcagtgggtggataaagggatttatctaccacagaatgggtttcatcaagctgcgtatctggatatgacccctacatttcaccagggcttccctgttgttgccaacagaaaccttcaggctgtgaccagtgcacaccaggcttacagcagtggccagacttatgggaatgggcagggcctgtatccgttcatggctgtggaggatgagggcttctttggaaaaggtggaacaagctgcaacacccagcaggccatgggtttattaagtcaacagatgaacttctatcatggctactctaccaatgtggattatgacagcttgcaggcagaagatacctacagcttccagagcacctctgatagtatcacacagttctcgagctctcctgtacggcatcagtaccaggccccttggcataccctggggacccagaatggttatgagacttag

SEQ ID NO: 2 - protein, 420 aa, Gallus gallus
MSAHLAMPSYGSVRCGHYYWPSPGSMDSASAAEAPAADLSLTTEQKTPCHPARHPQCSLSARRLLLKAARSQGAMAGPCRGVVGSTSHVSHWELSGGQGLGAAAGHAAKVLARPSCTTGNSAGLSCRRSLLPVLTLAAESAAVSSAPESEIISRSCTSKFLSDASPASSSSGTLIQYTPDSATSPTADHPSHRPTFQKVKDKGESGTRKAKSRTAFSQEQLQTLHQRFQSQKYLSPHQIRELAAALGLTYKQVKTWFQNQRMKFKRCQKESQWVDKGIYLPQNGFHQAAYLDMTPTFHQGFPVVANRNLQAVTSAHQAYSSGQTYGNGQGLYPFMAVEDEGFFGKGGTSCNTQQAMGLLSQQMNFYHGYSTNVDYDSLQAEDTYSFQSTSDSITQFSSSPVRHQYQAPWHTLGTQNGYET

SEQ ID NO: 3 - DNA, 1008 bp, Gallus gallus
atgaagcgggactcgacctgcatggaagatgaccgggttgatcagcgaacctgcctgatctgcggggacagagctacgggtctgcactatggcatcatctcctgcgagggctgcaaggggtttttcaaaaggagcatttgcaacgagcgggtttacagatgcagccgagacaagaactgtgtcatgtcacgcaagcagcgcaacagatgccagtactgccggctgctcaaatgcctccagatggggatgaaccggaaaggtgacactctgggcaccggcatgtcggcgtcgcacaccagttctggtgacacttttcttgcttttcaagtgccttgtcagattgtgtcattctgtgttttacttttatgccttcatattaactgtgcctgcaggtataaagtgactcaggcagagctgtttgcattgttgtgtcgtctggcggatgaattgcttttcaggcagattgcttggatcaagaagctgccattcttctgtgaactctcaatcaaggactatacctgcctgctcagctccacctggcaggagctgatcctgctttcctcgctgactgtttacagcaaacagatctttggtgacctcgcagacgtcacctccaagtactcaccctctgatgatgagctgcacagattcagtgaagagggcatggaggtgatggagcggttgatctacctctttcgcaaattcaaccagttgaaggtcagcaacgaggagtacgcatgcatgaaagccattaacttcctaaatcaagacattaggggtttaaccaatgcatcccaactggagcagctgaataagcggtactggtatgtttgccaggacttcacagagtacaaatacccacaccagccaaaccgttttccggatttaatgatgtgtttgccggagatacgatatattgcaggaaagatggtgaatgtcccgctggagcagctgccgctcctttttaaggccgttctgcattcctgcaagacaagtgtgagcaaagag

SEQ ID NO: 4 - protein, 336 aa, Gallus gallus
MKRDSTCMEDDRVDQRTCLICGDRATGLHYGIISCEGCKGFFKRSICNERVYRCSRDKNCVMSRKQRNRCQYCRLLKCLQMGMNRKGDTLGTGMSASHTSSGDTFLAFQVPCQIVSFCVLLLCLHINCACRYKVTQAELFALLCRLADELLFRQIAWIKKLPFFCELSIKDYTCLLSSTWQELILLSSLTVYSKQIFGDLADVTSKYSPSDDELHRFSEEGMEVMERLIYLFRKFNQLKVSNEEYACMKAINFLNQDIRGLTNASQLEQLNKRYWYVCQDFTEYKYPHQPNRFPDLMMCLPEIRYIAGKMVNVPLEQLPLLFKAVLHSCKTSVSKE

SEQ ID NO: 5 - DNA, 21 bp, Artificial
  PCR primer
acatgtgccctcttattgcac

SEQ ID NO: 6 - DNA, 21 bp, Artificial
  PCR primer
aaactccacaccatcatcagc

SEQ ID NO: 7 - DNA, 21 bp, Artificial
  PCR primer
atctttctgcccgtaggtgtt

SEQ ID NO: 8 - DNA, 21 bp, Artificial
  PCR primer
tgctgcccacttctcatttat

SEQ ID NO: 9 - DNA, 21 bp, Artificial
  PCR primer
atccttcccacaccagagact

SEQ ID NO: 10 - DNA, 21 bp, Artificial
  PCR primer
gagctcatgtcagcttcatcc

SEQ ID NO: 11 - DNA, 21 bp, Artificial
  PCR primer
gcaaaactctcgggaaaatct

SEQ ID NO: 12 - DNA, 21 bp, Artificial
  PCR primer
gcatcttctggtgttgtccat

SEQ ID NO: 13 - DNA, 21 bp, Artificial
  PCR primer
ctcgacttcgacaaccagaag

SEQ ID NO: 14 - DNA, 21 bp, Artificial
  PCR primer
tgcatcacactgttctcaagg

SEQ ID NO: 15 - DNA, 21 bp, Artificial
  PCR primer
tccattttgctctgaactgct

SEQ ID NO: 16 - DNA, 21 bp, Artificial
  PCR primer
ctctgtttctgcggatagcac

SEQ ID NO: 17 - DNA, 21 bp, Artificial
  PCR primer
taaagaacctgaaggggagga

SEQ ID NO: 18 - DNA, 21 bp, Artificial
  PCR primer
ctcttctattcgtcgccactg

SEQ ID NO: 19 - DNA, 20 bp, Artificial
  PCR primer
tgcagttcaacaaccagctc

SEQ ID NO: 20 - DNA, 20 bp, Artificial
  PCR primer
gtcagttctgtgccgtgaga

SEQ ID NO: 21 - DNA, 21 bp, Artificial
  PCR primer
ctccccatattccgcttctac

SEQ ID NO: 22 - DNA, 21 bp, Artificial
  PCR primer
tcagctcctgtaggttctcca

SEQ ID NO: 23 - DNA, 21 bp, Artificial
  PCR primer
caggaataatcgggaatggat

SEQ ID NO: 24 - DNA, 21 bp, Artificial
  PCR primer
caggacatttccgaagtacca

SEQ ID NO: 25 - DNA, 20 bp, Artificial
  PCR primer
ttggcattcccaattctagg

SEQ ID NO: 26 - DNA, 20 bp, Artificial
  PCR primer
gaaaggccccttttggtaag

SEQ ID NO: 27 - DNA, 20 bp, Artificial
  PCR primer
aacaggtttcctggatgtcg

SEQ ID NO: 28 - DNA, 20 bp, Artificial
  PCR primer
tttggatccctctctggatg

SEQ ID NO: 29 - DNA, 21 bp, Artificial
  PCR primer
acacttgttcgcagagcagtt

SEQ ID NO: 30 - DNA, 21 bp, Artificial
  PCR primer
gccacctcaataagggttagg

SEQ ID NO: 31 - DNA, 21 bp, Artificial
  PCR primer
cagccaccatcaagtacaaca

SEQ ID NO: 32 - DNA, 21 bp, Artificial
  PCR primer
ctgctgggaataagtgctctg

SEQ ID NO: 33 - DNA, 21 bp, Artificial
  PCR primer
aaccgatacagtctgctggaa

SEQ ID NO: 34 - DNA, 21 bp, Artificial
  PCR primer
agttgtttggcaggaggatct

SEQ ID NO: 35 - DNA, 21 bp, Artificial
  PCR primer
gtgggctctaccacaaaatga

SEQ ID NO: 36 - DNA, 21 bp, Artificial
  PCR primer
tgcctttggtaatgtttttcg

SEQ ID NO: 37 - DNA, 21 bp, Artificial
  PCR primer
atggccagtagtccctctgtt

SEQ ID NO: 38 - DNA, 21 bp, Artificial
  PCR primer
caatgccgtagtatgcaggat

SEQ ID NO: 39 - DNA, 21 bp, Artificial
  PCR primer
cactatgacctttggcagagc

SEQ ID NO: 40 - DNA, 21 bp, Artificial
  PCR primer
aatgcaccattcaaaacaagc

SEQ ID NO: 41 - DNA, 21 bp, Artificial
  PCR primer
cctcctaaccaaggagcattc

SEQ ID NO: 42 - DNA, 21 bp, Artificial
  PCR primer
ctgagatggacctggatgaaa

SEQ ID NO: 43 - DNA, 21 bp, Artificial
  PCR primer
actaccctcaggggttcttca

SEQ ID NO: 44 - DNA, 21 bp, Artificial
  PCR primer
cctcaaacacaagacccaaaa

SEQ ID NO: 45 - DNA, 21 bp, Artificial
  PCR primer
ccgtggccgtctataagaaat

SEQ ID NO: 46 - DNA, 21 bp, Artificial
  PCR primer
ccagtgtaaggatggtgagga

SEQ ID NO: 47 - DNA, 21 bp, Artificial
  PCR primer
acctcgccagccataactaat

SEQ ID NO: 48 - DNA, 21 bp, Artificial
  PCR primer
tctccttcatggggatcttct

SEQ ID NO: 49 - DNA, 21 bp, Artificial
  PCR primer
tggaacagggactatcctcct

SEQ ID NO: 50 - DNA, 21 bp, Artificial
  PCR primer
gctccaaagctgaaagaaggt

SEQ ID NO: 51 - DNA, 21 bp, Artificial
  PCR primer
aagaaaacaaagccagcaaca

SEQ ID NO: 52 - DNA, 21 bp, Artificial
  PCR primer
atctcttgggctccctcatta

SEQ ID NO: 53 - DNA, 21 bp, Artificial
  PCR primer
atgcacaaaaagtggcaaatc

SEQ ID NO: 54 - DNA, 21 bp, Artificial
  PCR primer
agttcgggcacaatctacctt

SEQ ID NO: 55 - DNA, 21 bp, Artificial
  PCR primer
gttcccacatgcttgtcactt

SEQ ID NO: 56 - DNA, 21 bp, Artificial
  PCR primer
aaagatttggatgcgtttgtg

SEQ ID NO: 57 - DNA, 21 bp, Artificial
  PCR primer
ccggatgcagactcattgtat

SEQ ID NO: 58 - DNA, 21 bp, Artificial
  PCR primer
tcatctttgcaggcttcaagt

SEQ ID NO: 59 - DNA, 21 bp, Artificial
  PCR primer
aattcagtgggaaagcatgtg

SEQ ID NO: 60 - DNA, 21 bp, Artificial
  PCR primer
ttcagatttgcaaagggattg

SEQ ID NO: 61 - DNA, 21 bp, Artificial
  PCR primer
aacctatgatggacccaggac

SEQ ID NO: 62 - DNA, 21 bp, Artificial
  PCR primer
cagaataagcatcgccagaag

SEQ ID NO: 63 - DNA, 21 bp, Artificial
  PCR primer
taagcgtttagacccccttgt

SEQ ID NO: 64 - DNA, 21 bp, Artificial
  PCR primer
atgatccttcccttgtcgtct

SEQ ID NO: 65 - DNA, 21 bp, Artificial
  PCR primer
cagattgtgtgcaactcctca

SEQ ID NO: 66 - DNA, 21 bp, Artificial
  PCR primer
tcgacgttcttcacttttgct

SEQ ID NO: 67 - DNA, 21 bp, Artificial
  PCR primer
gtggaatagggaggagtctgg

SEQ ID NO: 68 - DNA, 21 bp, Artificial
  PCR primer
ggtgataaacacggaaagcaa

SEQ ID NO: 69 - DNA, 21 bp, Artificial
  PCR primer
tgccagtaccctacaaacctg

SEQ ID NO: 70 - DNA, 21 bp, Artificial
  PCR primer
gctacggctgctaatgcatac

SEQ ID NO: 71 - DNA, 21 bp, Artificial
  PCR primer
tgctgcagtatagggcttgtt

SEQ ID NO: 72 - DNA, 21 bp, Artificial
  PCR primer
gacgaggagtcagagtgcaac

SEQ ID NO: 73 - DNA, 21 bp, Artificial
  PCR primer
tgtatctgtcccgtgccttac

SEQ ID NO: 74 - DNA, 21 bp, Artificial
  PCR primer
ttttgagcttgcgttccttta

SEQ ID NO: 75 - DNA, 21 bp, Artificial
  PCR primer
tgcacgaagactctttccact

SEQ ID NO: 76 - DNA, 21 bp, Artificial
  PCR primer
caaagcagcaagaatcctgac

SEQ ID NO: 77 - DNA, 21 bp, Artificial
  PCR primer
gctgtctgtgctctgctcttt

SEQ ID NO: 78 - DNA, 21 bp, Artificial
  PCR primer
ttgaccccattcagttagcac

SEQ ID NO: 79 - DNA, 21 bp, Artificial
  PCR primer
atacctggttgctccgaaagt

SEQ ID NO: 80 - DNA, 21 bp, Artificial
  PCR primer
ccaccacatttcaagcagttt

SEQ ID NO: 81 - DNA, 21 bp, Artificial
  PCR primer
ccacataccactgaggctgtt

SEQ ID NO: 82 - DNA, 21 bp, Artificial
  PCR primer
accatgaactgcagtgtttcc

SEQ ID NO: 83 - DNA, 21 bp, Artificial
  PCR primer
tccctttgcatccacactatc

SEQ ID NO: 84 - DNA, 21 bp, Artificial
  PCR primer
gcttcaccaccatttttgaga

SEQ ID NO: 85 - DNA, 21 bp, Artificial
  PCR primer
gcttctgcaggtcatcatctc

SEQ ID NO: 86 - DNA, 21 bp, Artificial
  PCR primer
gcgttgagctatccctctctt

SEQ ID NO: 87 - DNA, 21 bp, Artificial
  PCR primer
caatcttggcagagtgagagg

SEQ ID NO: 88 - DNA, 21 bp, Artificial
  PCR primer
cgtcctcatggaggtaagaca

SEQ ID NO: 89 - DNA, 21 bp, Artificial
  PCR primer
ctggcacagatcttcaaggag

SEQ ID NO: 90 - DNA, 21 bp, Artificial
  PCR primer
acatcacgaccagttggagac

SEQ ID NO: 91 - DNA, 21 bp, Artificial
  PCR primer
caaggtgtacgactccatgct

SEQ ID NO: 92 - DNA, 21 bp, Artificial
  PCR primer
gtagaaatcccggatgatggt

SEQ ID NO: 93 - DNA, 21 bp, Artificial
  PCR primer
ttacagctagcacggacgttt

SEQ ID NO: 94 - DNA, 21 bp, Artificial
  PCR primer
ttggaaaccaagtcattctgg

SEQ ID NO: 95 - DNA, 21 bp, Artificial
  PCR primer
ggcggagtaaccctatctgtc

SEQ ID NO: 96 - DNA, 21 bp, Artificial
  PCR primer
gtggtatccacagctgcattt

SEQ ID NO: 97 - DNA, 21 bp, Artificial
  PCR primer
catctgttggagctcttcctg

SEQ ID NO: 98 - DNA, 21 bp, Artificial
  PCR primer
ccaaagcagtccaggtaatca

SEQ ID NO: 99 - DNA, 21 bp, Artificial
  PCR primer
attgctggcaagaatagctga

SEQ ID NO: 100 - DNA, 21 bp, Artificial
  PCR primer
tgaaaagagacctccagcaaa

SEQ ID NO: 101 - DNA, 21 bp, Artificial
  PCR primer
gtttgccaggacttcacagag

SEQ ID NO: 102 - DNA, 20 bp, Artificial
  PCR primer
cgggacattcaccatctttc

SEQ ID NO: 103 - DNA, 21 bp, Artificial
  PCR primer
gctctggagtggatgaacttg

SEQ ID NO: 104 - DNA, 21 bp, Artificial
  PCR primer
gtgcacctcaatctggttgtt

SEQ ID NO: 105 - DNA, 21 bp, Artificial
  PCR primer
aggactcgagcaaggagtttc

SEQ ID NO: 106 - DNA, 21 bp, Artificial
  PCR primer
gtctgttgagctgagcgaact

SEQ ID NO: 107 - DNA, 21 bp, Artificial
  PCR primer
tctggactgcaaagagcattt

SEQ ID NO: 108 - DNA, 21 bp, Artificial
  PCR primer
gctcaaaccaaaccaaaacaa

SEQ ID NO: 109 - DNA, 21 bp, Artificial
  PCR primer
cgaagcccttgagaaagagtt

SEQ ID NO: 110 - DNA, 21 bp, Artificial
  PCR primer
actactgctgatggggatgtg

SEQ ID NO: 111 - DNA, 21 bp, Artificial
  PCR primer
ctccacttcaaatgccagtgt

SEQ ID NO: 112 - DNA, 21 bp, Artificial
  PCR primer
gccaaacatggcttgataaaa

SEQ ID NO: 113 - DNA, 21 bp, Artificial
  PCR primer
tgttcgcttccgagtcttaaa

SEQ ID NO: 114 - DNA, 21 bp, Artificial
  PCR primer
cctttccgtaggaacaaaacc

SEQ ID NO: 115 - DNA, 21 bp, Artificial
  PCR primer
tgcagggacagaagaagaaga

SEQ ID NO: 116 - DNA, 21 bp, Artificial
  PCR primer
tgtgacccataatcccaacat

SEQ ID NO: 117 - DNA, 21 bp, Artificial
  PCR primer
agagtgaggctgcacaacaac

SEQ ID NO: 118 - DNA, 21 bp, Artificial
  PCR primer
atgagagtggccccatagaac

SEQ ID NO: 119 - DNA, 21 bp, Artificial
  PCR primer
ataacatccagccgtcaagtg

SEQ ID NO: 120 - DNA, 21 bp, Artificial
  PCR primer
agtttttgcttctgggatggt

SEQ ID NO: 121 - DNA, 21 bp, Artificial
  PCR primer
gagcagaaccactacgacagc

SEQ ID NO: 122 - DNA, 21 bp, Artificial
  PCR primer
cctggaactggtcataggtga

SEQ ID NO: 123 - DNA, 21 bp, Artificial
  PCR primer
gacgatccagagttcagcaac

SEQ ID NO: 124 - DNA, 21 bp, Artificial
  PCR primer
aggttgtttttgctggtgatg

SEQ ID NO: 125 - DNA, 21 bp, Artificial
  PCR primer
tgtgcttagggaatgtgaagg

SEQ ID NO: 126 - DNA, 21 bp, Artificial
  PCR primer
caatgcttttcacactcagca

SEQ ID NO: 127 - DNA, 21 bp, Artificial
  PCR primer
ggacgtttccattcagacaaa

SEQ ID NO: 128 - DNA, 21 bp, Artificial
  PCR primer
agctttgagtcattgcgttgt

SEQ ID NO: 129 - DNA, 21 bp, Artificial
  PCR primer
acgtggtgttcctctacatgc

SEQ ID NO: 130 - DNA, 21 bp, Artificial
  PCR primer
gaaggcctccttacagctctc

SEQ ID NO: 131 - DNA, 19 bp, Artificial
  PCR primer
agatctgcaaacccgtgct

SEQ ID NO: 132 - DNA, 21 bp, Artificial
  PCR primer
tcgagaaacaaccttctgctc

SEQ ID NO: 133 - DNA, 21 bp, Artificial
  PCR primer
aaaacccagcatacacactgc

SEQ ID NO: 134 - DNA, 21 bp, Artificial
  PCR primer
aaggttcaccttcttcgggta

SEQ ID NO: 135 - DNA, 21 bp, Artificial
  PCR primer
cgtctgatttggttgtggact

SEQ ID NO: 136 - DNA, 21 bp, Artificial
  PCR primer
ttcatggctgaccctacaaac

SEQ ID NO: 137 - DNA, 21 bp, Artificial
  PCR primer
caccttctacccgtggctaat

SEQ ID NO: 138 - DNA, 21 bp, Artificial
  PCR primer
cgcttgaactttgtccttctg

SEQ ID NO: 139 - DNA, 21 bp, Artificial
  PCR primer
gagaagaacgccacacacttc

SEQ ID NO: 140 - DNA, 21 bp, Artificial
  PCR primer
tgttgacctccagctctgtct

SEQ ID NO: 141 - DNA, 21 bp, Artificial
  PCR primer
atcagactacccatccccatc

SEQ ID NO: 142 - DNA, 21 bp, Artificial
  PCR primer
gggagtcttctcctgtcatcc

SEQ ID NO: 143 - DNA, 21 bp, Artificial
  PCR primer
ctcaccatgtggatgaaggat

SEQ ID NO: 144 - DNA, 21 bp, Artificial
  PCR primer
acgatgagggtgtagttcacg

SEQ ID NO: 145 - DNA, 21 bp, Artificial
  PCR primer
gtctctctcactcgggctttt

SEQ ID NO: 146 - DNA, 21 bp, Artificial
  PCR primer
tgtctgaggactgagctggat

SEQ ID NO: 147 - DNA, 21 bp, Artificial
  PCR primer
gtgctatccctacccaaaagc

SEQ ID NO: 148 - DNA, 21 bp, Artificial
  PCR primer
ggcttggctgtcaacatacat

SEQ ID NO: 149 - DNA, 21 bp, Artificial
  PCR primer
tgagaaaagagggcattcaga

SEQ ID NO: 150 - DNA, 21 bp, Artificial
  PCR primer
gcaggatgaattgaagatcca

SEQ ID NO: 151 - DNA, 21 bp, Artificial
  PCR primer
gcagaaccacagggtatcaga

SEQ ID NO: 152 - DNA, 21 bp, Artificial
  PCR primer
tcgagcacacattcccttaac

SEQ ID NO: 153 - DNA, 21 bp, Artificial
  PCR primer
cccttttgcaaggagaatacc

SEQ ID NO: 154 - DNA, 21 bp, Artificial
  PCR primer
gctggtctctgtccatgtgat

SEQ ID NO: 155 - DNA, 21 bp, Artificial
  PCR primer
cagtatggtgctggtgtttgg

SEQ ID NO: 156 - DNA, 21 bp, Artificial
  PCR primer
catacatgtgtcccccactct

SEQ ID NO: 157 - DNA, 21 bp, Artificial
  PCR primer
cacgctcacaggacagtgtta

SEQ ID NO: 158 - DNA, 21 bp, Artificial
  PCR primer
gttgtcatcagatgcctggat

SEQ ID NO: 159 - DNA, 21 bp, Artificial
  PCR primer
gggacagggaaatctgaagag

SEQ ID NO: 160 - DNA, 21 bp, Artificial
  PCR primer
ccttgcgtgtaatttttgcat

SEQ ID NO: 161 - DNA, 21 bp, Artificial
  PCR primer
taccactggagccaatttcac

SEQ ID NO: 162 - DNA, 21 bp, Artificial
  PCR primer
cagggggaacttattcagagg

SEQ ID NO: 163 - DNA, 21 bp, Artificial
  PCR primer
gatgggtttgctgagatgtgt

SEQ ID NO: 164 - DNA, 21 bp, Artificial
  PCR primer
gcccctttagagaccattttg

SEQ ID NO: 165 - DNA, 21 bp, Artificial
  PCR primer
atgcccagtctaggaaatcgt

SEQ ID NO: 166 - DNA, 21 bp, Artificial
  PCR primer
ctccagtgtgggttctgatgt

SEQ ID NO: 167 - DNA, 21 bp, Artificial
  PCR primer
acatcaacaccaagtgcatca

SEQ ID NO: 168 - DNA, 21 bp, Artificial
  PCR primer
ggcgtactcacacatctccat

SEQ ID NO: 169 - DNA, 21 bp, Artificial
  PCR primer
acacccagctgttcaccatag

SEQ ID NO: 170 - DNA, 21 bp, Artificial
  PCR primer
ctgaagcaggagcagctctaa

SEQ ID NO: 171 - DNA, 21 bp, Artificial
  PCR primer
tcaacgagttcgaccaatacc

SEQ ID NO: 172 - DNA, 21 bp, Artificial
  PCR primer
gctcagctgctctgttttgat

SEQ ID NO: 173 - DNA, 21 bp, Artificial
  PCR primer
tggaggaagtcagggaagaat

SEQ ID NO: 174 - DNA, 21 bp, Artificial
  PCR primer
agatctggggatgaggtgtct

SEQ ID NO: 175 - DNA, 21 bp, Artificial
  PCR primer
gacaagcaggattacccacaa

SEQ ID NO: 176 - DNA, 21 bp, Artificial
  PCR primer
ctgggcatcgaacattaggta

SEQ ID NO: 177 - DNA, 21 bp, Artificial
  PCR primer
gtcaggtagccacaagaccag

SEQ ID NO: 178 - DNA, 21 bp, Artificial
  PCR primer
gaccatgccctgtcttcagta

SEQ ID NO: 179 - DNA, 21 bp, Artificial
  PCR primer
cttgacgtagcagagggaaaa

SEQ ID NO: 180 - DNA, 21 bp, Artificial
  PCR primer
aagctttgcagagtcaagtgc

SEQ ID NO: 181 - DNA, 21 bp, Artificial
  PCR primer
gctggacccttctctctctgt

SEQ ID NO: 182 - DNA, 21 bp, Artificial
  PCR primer
ccgttacagcgagatgtcatt

SEQ ID NO: 183 - DNA, 21 bp, Artificial
  PCR primer
aaccgtctcattgacacaagg

SEQ ID NO: 184 - DNA, 20 bp, Artificial
  PCR primer
accttcaggctttcttgctt

SEQ ID NO: 185 - DNA, 21 bp, Artificial
  PCR primer
atccaggggtcaggaagtcta

SEQ ID NO: 186 - DNA, 21 bp, Artificial
  PCR primer
acccttgaggcagtttttcat

SEQ ID NO: 187 - DNA, 21 bp, Artificial
  PCR primer
caaaagaaagagcagccaatg

SEQ ID NO: 188 - DNA, 21 bp, Artificial
  PCR primer
cccatcgtacttgcaggagta

SEQ ID NO: 189 - DNA, 21 bp, Artificial
  PCR primer
aatgagcaccctcttcgaaat

SEQ ID NO: 190 - DNA, 21 bp, Artificial
  PCR primer
tgacgctctggacctcttaaa

SEQ ID NO: 191 - DNA, 21 bp, Artificial
  PCR primer
aggccgaggaagaaattatga

SEQ ID NO: 192 - DNA, 21 bp, Artificial
  PCR primer
actgcaggaggtggtattcct

SEQ ID NO: 193 - DNA, 21 bp, Artificial
  PCR primer
gccaagagaaaaaggcagagt

SEQ ID NO: 194 - DNA, 21 bp, Artificial
  PCR primer
tccgttgaggacacatcctac

SEQ ID NO: 195 - DNA, 21 bp, Artificial
  PCR primer
cccagtcctgtcctgttcata

SEQ ID NO: 196 - DNA, 21 bp, Artificial
  PCR primer
cggtctcgtacgtctaacagc

SEQ ID NO: 197 - DNA, 21 bp, Artificial
  PCR primer
acaaattggaccatgtggtgt

SEQ ID NO: 198 - DNA, 21 bp, Artificial
  PCR primer
gtccttatactgccggtcctc

SEQ ID NO: 199 - DNA, 21 bp, Artificial
  PCR primer
ttctttgagccagtctttgga

SEQ ID NO: 200 - DNA, 21 bp, Artificial
  PCR primer
cagggggtccttaaacaagag

SEQ ID NO: 201 - DNA, 21 bp, Artificial
  PCR primer
gccacctccgtcctttactac

SEQ ID NO: 202 - DNA, 21 bp, Artificial
  PCR primer
tttacatccacgtctgggaag

SEQ ID NO: 203 - DNA, 21 bp, Artificial
  PCR primer
tcttctacaagcagcccatgt

SEQ ID NO: 204 - DNA, 21 bp, Artificial
  PCR primer
gtcctcaatgacaccgacagt

SEQ ID NO: 205 - DNA, 21 bp, Artificial
  PCR primer
atggacgaaatgcctgtagtg

SEQ ID NO: 206 - DNA, 21 bp, Artificial
  PCR primer
tgacgtccagtttttccattc

SEQ ID NO: 207 - DNA, 21 bp, Artificial
  PCR primer
ccccttgtcagttacaggtca

SEQ ID NO: 208 - DNA, 21 bp, Artificial
  PCR primer
gtttccttggaatttgccttc

SEQ ID NO: 209 - DNA, 21 bp, Artificial
  PCR primer
gactgctttgtcaacctcagc

SEQ ID NO: 210 - DNA, 21 bp, Artificial
  PCR primer
gctgttgcagaagatgaggac

SEQ ID NO: 211 - DNA, 20 bp, Artificial
  PCR primer
cactgacaaccctgatggtg

SEQ ID NO: 212 - DNA, 20 bp, Artificial
  PCR primer
tggcactggcatatgtagga

SEQ ID NO: 213 - DNA, 21 bp, Artificial
  PCR primer
ttgaccaaatttccattgagg

SEQ ID NO: 214 - DNA, 21 bp, Artificial
  PCR primer
acagaagaaccccagaccagt

SEQ ID NO: 215 - DNA, 20 bp, Artificial
  PCR primer
caccagtcaggacccaaagt

SEQ ID NO: 216 - DNA, 20 bp, Artificial
  PCR primer
ggggatgaaacctttttggt

SEQ ID NO: 217 - DNA, 21 bp, Artificial
  PCR primer
gggacttcctcaagtggtagg

SEQ ID NO: 218 - DNA, 21 bp, Artificial
  PCR primer
acgtggaaggaagaagtggat

SEQ ID NO: 219 - DNA, 21 bp, Artificial
  PCR primer
acaagaaaatcaatgcgatgg

SEQ ID NO: 220 - DNA, 21 bp, Artificial
  PCR primer
gttgttctccagcacgatctc

SEQ ID NO: 221 - DNA, 21 bp, Artificial
  PCR primer
ccagagaggcctagaaaggaa

SEQ ID NO: 222 - DNA, 21 bp, Artificial
  PCR primer
tcctctgctgcactgaagatt

SEQ ID NO: 223 - DNA, 21 bp, Artificial
  PCR primer
aaacgagacagctccatcaga

SEQ ID NO: 224 - DNA, 21 bp, Artificial
  PCR primer
ctgccctaagtttacccaagg

SEQ ID NO: 225 - DNA, 21 bp, Artificial
  PCR primer
acaagaacgacgtcatgatcc

SEQ ID NO: 226 - DNA, 21 bp, Artificial
  PCR primer
cgtggtaaaggagatgtgctc

SEQ ID NO: 227 - DNA, 20 bp, Artificial
  PCR primer
agctgcacgtcgaagttttt

SEQ ID NO: 228 - DNA, 20 bp, Artificial
  PCR primer
ttccttgtcccactctcacc

SEQ ID NO: 229 - DNA, 21 bp, Artificial
  PCR primer
acatgatggtggactttgagc

SEQ ID NO: 230 - DNA, 21 bp, Artificial
  PCR primer
gtacagcatggagaggggact

SEQ ID NO: 231 - DNA, 21 bp, Artificial
  PCR primer
gtcttcgatgccatgttcaat

SEQ ID NO: 232 - DNA, 21 bp, Artificial
  PCR primer
gtctgctcggaggttcttctt

SEQ ID NO: 233 - DNA, 21 bp, Artificial
  PCR primer
agcaagcagcaaagcagatag

SEQ ID NO: 234 - DNA, 21 bp, Artificial
  PCR primer
aactgaaccgtcctttgacct

SEQ ID NO: 235 - DNA, 21 bp, Artificial
  PCR primer
taacccagtcggattgctatg

SEQ ID NO: 236 - DNA, 21 bp, Artificial
  PCR primer
ctggattttggagacatcgaa

SEQ ID NO: 237 - DNA, 21 bp, Artificial
  PCR primer
ccaagaccaaatgcaagtcat

SEQ ID NO: 238 - DNA, 21 bp, Artificial
  PCR primer
aatgcactgcaatgtttttcc

SEQ ID NO: 239 - DNA, 22 bp, Artificial
  PCR primer
tacctaactggcatagagacct

SEQ ID NO: 240 - DNA, 19 bp, Artificial
  PCR primer
tcctgtggaagacaatgga

SEQ ID NO: 241 - DNA, 21 bp, Artificial
  PCR primer
tttcagctcttggacctttca

SEQ ID NO: 242 - DNA, 21 bp, Artificial
  PCR primer
ttttcaagttcttccgcttga

SEQ ID NO: 243 - DNA, 21 bp, Artificial
  PCR primer
gtttctgcgacacctgagaag

SEQ ID NO: 244 - DNA, 21 bp, Artificial
  PCR primer
ttcacatgcagcaagattgag

SEQ ID NO: 245 - DNA, 21 bp, Artificial
  PCR primer
gcagagaaaagggaaaaagga

SEQ ID NO: 246 - DNA, 21 bp, Artificial
  PCR primer
tttcctagggaggggtatgaa

SEQ ID NO: 247 - DNA, 21 bp, Artificial
  PCR primer
tagatggagagcgtcaggaaa

SEQ ID NO: 248 - DNA, 21 bp, Artificial
  PCR primer
catgactcaggaacacggttt

SEQ ID NO: 249 - DNA, 21 bp, Artificial
  PCR primer
caacgcatccctaatttctga

SEQ ID NO: 250 - DNA, 21 bp, Artificial
  PCR primer
acggacagaccaaaagaggtt

SEQ ID NO: 251 - DNA, 20 bp, Artificial
  PCR primer
gggaaatgatggacaaaacg

SEQ ID NO: 252 - DNA, 20 bp, Artificial
  PCR primer
ggcagtcgtttcaagagagg

SEQ ID NO: 253 - DNA, 21 bp, Artificial
  PCR primer
cccaatagaaggggcatagag

SEQ ID NO: 254 - DNA, 21 bp, Artificial
  PCR primer
cttggtaactgcgggaataca

SEQ ID NO: 255 - DNA, 21 bp, Artificial
  PCR primer
tctcctcaacctgaagaacga

SEQ ID NO: 256 - DNA, 21 bp, Artificial
  PCR primer
cttggcaaccatttgaggtaa

SEQ ID NO: 257 - DNA, 21 bp, Artificial
  PCR primer
gtgcagacaatggaggacatt

SEQ ID NO: 258 - DNA, 21 bp, Artificial
  PCR primer
tcttggttgttggtttcttgg

SEQ ID NO: 259 - DNA, 21 bp, Artificial
  PCR primer
tcaggattcttgctgctttgt

SEQ ID NO: 260 - DNA, 21 bp, Artificial
  PCR primer
ggatggtagagcccttggtag

SEQ ID NO: 261 - DNA, 21 bp, Artificial
  PCR primer
ccaagcagctacgattctgtc

SEQ ID NO: 262 - DNA, 21 bp, Artificial
  PCR primer
ggattccctggtagctttgtc

SEQ ID NO: 263 - DNA, 21 bp, Artificial
  PCR primer
ggctggtttgtgtttgacatt

SEQ ID NO: 264 - DNA, 21 bp, Artificial
  PCR primer
ccatgaatggctgttttgact

SEQ ID NO: 265 - DNA, 21 bp, Artificial
  PCR primer
aggagaagagcacactgacca

SEQ ID NO: 266 - DNA, 21 bp, Artificial
  PCR primer
gtgtccagatagctgccaatg

SEQ ID NO: 267 - DNA, 21 bp, Artificial
  PCR primer
cagtgcaaagacctgaggaag

SEQ ID NO: 268 - DNA, 21 bp, Artificial
  PCR primer
cttcagtcaggatggaagcag

SEQ ID NO: 269 - DNA, 21 bp, Artificial
  PCR primer
ccttgccaacatctctctctg

SEQ ID NO: 270 - DNA, 21 bp, Artificial
  PCR primer
caagcgagagtccattttctg

SEQ ID NO: 271 - DNA, 21 bp, Artificial
  PCR primer
tcttattactggagcgcctga

SEQ ID NO: 272 - DNA, 21 bp, Artificial
  PCR primer
caggatggagagagtttgctg

SEQ ID NO: 273 - DNA, 21 bp, Artificial
  PCR primer
ctggaagagcttggatgtctg

SEQ ID NO: 274 - DNA, 21 bp, Artificial
  PCR primer
tcctctgcctgtcttactgga

SEQ ID NO: 275 - DNA, 21 bp, Artificial
  PCR primer
atctgattgcacagagccagt

SEQ ID NO: 276 - DNA, 21 bp, Artificial
  PCR primer
accctgatgtcattgcagaac

SEQ ID NO: 277 - DNA, 21 bp, Artificial
  PCR primer
ggacctggaacaggttttctc

SEQ ID NO: 278 - DNA, 21 bp, Artificial
  PCR primer
agcaactgcatgtccattacc

SEQ ID NO: 279 - DNA, 21 bp, Artificial
  PCR primer
tcaggaaagccagaacagaaa

SEQ ID NO: 280 - DNA, 21 bp, Artificial
  PCR primer
agacagctgaggaaggtgtga

SEQ ID NO: 281 - DNA, 21 bp, Artificial
  PCR primer
acacttgttcgcagagcagtt

SEQ ID NO: 282 - DNA, 21 bp, Artificial
  PCR primer
gccacctcaataagggttagg

SEQ ID NO: 283 - DNA, 21 bp, Artificial
  PCR primer
cctgaatgctcttttcagtgc

SEQ ID NO: 284 - DNA, 21 bp, Artificial
  PCR primer
cgcttctcaccactgacctac

SEQ ID NO: 285 - DNA, 21 bp, Artificial
  PCR primer
tcagacctgatccagagccta

SEQ ID NO: 286 - DNA, 21 bp, Artificial
  PCR primer
cattgcaacaccagtgacatc

SEQ ID NO: 287 - DNA, 21 bp, Artificial
  PCR primer
gcgaaggagaggctcaatact

SEQ ID NO: 288 - DNA, 21 bp, Artificial
  PCR primer
ggctgtgattccacgataaaa

SEQ ID NO: 289 - DNA, 21 bp, Artificial
  PCR primer
atcaataccctggaacccttg

SEQ ID NO: 290 - DNA, 21 bp, Artificial
  PCR primer
gctcatcagcatcaaatccat

SEQ ID NO: 291 - DNA, 20 bp, Artificial
  PCR primer
ggtgtgcggagataaggtgt

SEQ ID NO: 292 - DNA, 20 bp, Artificial
  PCR primer
gctcgcacagcttctagctt

SEQ ID NO: 293 - DNA, 21 bp, Artificial
  PCR primer
attttgcaggtgggcttaact

SEQ ID NO: 294 - DNA, 21 bp, Artificial
  PCR primer
aaacatggccccattctaaac

SEQ ID NO: 295 - DNA, 21 bp, Artificial
  PCR primer
gccggtatgaccagtacaaga

SEQ ID NO: 296 - DNA, 21 bp, Artificial
  PCR primer
cttgaagctcctgttcctcct

SEQ ID NO: 297 - DNA, 21 bp, Artificial
  PCR primer
aacaacaaaatggtggagtgc

SEQ ID NO: 298 - DNA, 21 bp, Artificial
  PCR primer
ggtgcaatgccagtgtaactt

SEQ ID NO: 299 - DNA, 21 bp, Artificial
  PCR primer
aacgaggaggagctgaaaaag

SEQ ID NO: 300 - DNA, 21 bp, Artificial
  PCR primer
tgtagggaatgttgaggttgc

SEQ ID NO: 301 - DNA, 21 bp, Artificial
  PCR primer
agccctgatcatcctcttgat

SEQ ID NO: 302 - DNA, 21 bp, Artificial
  PCR primer
cctggtcctcagtctttaccc

SEQ ID NO: 303 - DNA, 21 bp, Artificial
  PCR primer
gcatttctttgcttgtgcttt

SEQ ID NO: 304 - DNA, 21 bp, Artificial
  PCR primer
cctggggtcgtatttttcttc

SEQ ID NO: 305 - DNA, 20 bp, Artificial
  PCR primer
ctgctctggccatagtgaca

SEQ ID NO: 306 - DNA, 20 bp, Artificial
  PCR primer
cagcaattgactgctttcca

SEQ ID NO: 307 - DNA, 21 bp, Artificial
  PCR primer
ctggagcggtacaacagagag

SEQ ID NO: 308 - DNA, 21 bp, Artificial
  PCR primer
ggcaaagctcatgcttatctg

SEQ ID NO: 309 - DNA, 21 bp, Artificial
  PCR primer
aatcctccgggtaccactatg

SEQ ID NO: 310 - DNA, 21 bp, Artificial
  PCR primer
tcttcttattgcggtcattgc

SEQ ID NO: 311 - DNA, 21 bp, Artificial
  PCR primer
tctacaaaccgtgcttcgtct

SEQ ID NO: 312 - DNA, 21 bp, Artificial
  PCR primer
tcctccttcacctccttcttc

SEQ ID NO: 313 - DNA, 21 bp, Artificial
  PCR primer
tgaacgaagacatccgtatcc

SEQ ID NO: 314 - DNA, 21 bp, Artificial
  PCR primer
ctccagaagtgctcaaggatg

SEQ ID NO: 315 - DNA, 21 bp, Artificial
  PCR primer
cccaactaaaaccaacgttca

SEQ ID NO: 316 - DNA, 21 bp, Artificial
  PCR primer
tgaactgtttttcggccatag

SEQ ID NO: 317 - DNA, 20 bp, Artificial
  PCR primer
cagcgaagatgacaagtgga

SEQ ID NO: 318 - DNA, 20 bp, Artificial
  PCR primer
tatccgtcaacttccgaacc

SEQ ID NO: 319 - DNA, 21 bp, Artificial
  PCR primer
acagcaagtacttcggcaaga

SEQ ID NO: 320 - DNA, 21 bp, Artificial
  PCR primer
actgctgcgacttctcatagc

SEQ ID NO: 321 - DNA, 21 bp, Artificial
  PCR primer
catggagagaaacagggttca

SEQ ID NO: 322 - DNA, 21 bp, Artificial
  PCR primer
cacctcccacagcacataact

SEQ ID NO: 323 - DNA, 20 bp, Artificial
  PCR primer
accgcttgtccaccagttac

SEQ ID NO: 324 - DNA, 20 bp, Artificial
  PCR primer
ggcaacgcaggaatttatgt

SEQ ID NO: 325 - DNA, 21 bp, Artificial
  PCR primer
tgtctgcatagactgcaccac

SEQ ID NO: 326 - DNA, 21 bp, Artificial
  PCR primer
tagtgtgcacatgcctacagc

SEQ ID NO: 327 - DNA, 21 bp, Artificial
  PCR primer
ctgataagtgccatccgttgt

SEQ ID NO: 328 - DNA, 21 bp, Artificial
  PCR primer
ggtttttcaacatgctttgga

SEQ ID NO: 329 - DNA, 21 bp, Artificial
  PCR primer
tggagatcctcaaagtcatgg

SEQ ID NO: 330 - DNA, 21 bp, Artificial
  PCR primer
gtcccttacacttcccaaagc

SEQ ID NO: 331 - DNA, 21 bp, Artificial
  PCR primer
gaaggaagcacaggaagctct

SEQ ID NO: 332 - DNA, 21 bp, Artificial
  PCR primer
gcatgtcatccatgttcactg

SEQ ID NO: 333 - DNA, 21 bp, Artificial
  PCR primer
ggaccaataagatggctctgc

SEQ ID NO: 334 - DNA, 21 bp, Artificial
  PCR primer
atgagcgtgtcctcatacagg

SEQ ID NO: 335 - DNA, 20 bp, Artificial
  PCR primer
acctggagatcttccccaac

SEQ ID NO: 336 - DNA, 20 bp, Artificial
  PCR primer
cccttgtgagcttcttcgac

SEQ ID NO: 337 - DNA, 21 bp, Artificial
  PCR primer
atgaacatgtcctacgccaac

SEQ ID NO: 338 - DNA, 21 bp, Artificial
  PCR primer
gaggttggattgcccatagat

SEQ ID NO: 339 - DNA, 21 bp, Artificial
  PCR primer
tctggaggagaacacagagga

SEQ ID NO: 340 - DNA, 21 bp, Artificial
  PCR primer
gaactgtgagccattggtgtt

SEQ ID NO: 341 - DNA, 21 bp, Artificial
  PCR primer
gatgcgttaacacgggatcta

SEQ ID NO: 342 - DNA, 21 bp, Artificial
  PCR primer
ttcaggagcagccaaagttta

SEQ ID NO: 343 - DNA, 21 bp, Artificial
  PCR primer
gatgatgggaactcagccata

SEQ ID NO: 344 - DNA, 21 bp, Artificial
  PCR primer
tccaatacgccagtttggtag

SEQ ID NO: 345 - DNA, 21 bp, Artificial
  PCR primer
gacttccttatgctccccatc

SEQ ID NO: 346 - DNA, 21 bp, Artificial
  PCR primer
ggaaaaggtagcttccctcct

SEQ ID NO: 347 - DNA, 21 bp, Artificial
  PCR primer
tagagcagagatggtggcact

SEQ ID NO: 348 - DNA, 21 bp, Artificial
  PCR primer
aagttgtgggtgtgagtgagg

SEQ ID NO: 349 - DNA, 21 bp, Artificial
  PCR primer
atctttcacccttgcattgtg

SEQ ID NO: 350 - DNA, 21 bp, Artificial
  PCR primer
ggttcccactgtcaagaaaca

SEQ ID NO: 351 - DNA, 21 bp, Artificial
  PCR primer
gagctcctgttgtcaaacgtc

SEQ ID NO: 352 - DNA, 21 bp, Artificial
  PCR primer
agccataaacagcagaaagca

SEQ ID NO: 353 - DNA, 21 bp, Artificial
  PCR primer
gtccagtttgttggtgaagga

SEQ ID NO: 354 - DNA, 21 bp, Artificial
  PCR primer
gggaggagagagtcgttatgg

SEQ ID NO: 355 - DNA, 21 bp, Artificial
  PCR primer
aatccctttgtgaccaattcc

SEQ ID NO: 356 - DNA, 21 bp, Artificial
  PCR primer
tctactccaaggaggggaaaa

SEQ ID NO: 357 - DNA, 21 bp, Artificial
  PCR primer
gtcggctgtactctgtccaag

SEQ ID NO: 358 - DNA, 21 bp, Artificial
  PCR primer
cgacttgatgaagatggggta

SEQ ID NO: 359 - DNA, 21 bp, Artificial
  PCR primer
actggaaagggctctaagctg

SEQ ID NO: 360 - DNA, 21 bp, Artificial
  PCR primer
caacattccagtggcttcaat

SEQ ID NO: 361 - DNA, 21 bp, Artificial
  PCR primer
gctgacttggaagcagacaac

SEQ ID NO: 362 - DNA, 21 bp, Artificial
  PCR primer
tccaggaactgctcaagaaga

SEQ ID NO: 363 - DNA, 21 bp, Artificial
  PCR primer
ctccttgctggatgagaacag

SEQ ID NO: 364 - DNA, 21 bp, Artificial
  PCR primer
gtgctccagcaggtacatcat

SEQ ID NO: 365 - DNA, 21 bp, Artificial
  PCR primer
ggaacagctgctaaacgtcac

SEQ ID NO: 366 - DNA, 21 bp, Artificial
  PCR primer
tctagggttctcgcttccttc

SEQ ID NO: 367 - DNA, 21 bp, Artificial
  PCR primer
ctccatgcactctctctacgg

SEQ ID NO: 368 - DNA, 21 bp, Artificial
  PCR primer
tcaccaggaaatatgctaccg

SEQ ID NO: 369 - DNA, 21 bp, Artificial
  PCR primer
ggcatttctccatttctttcc

SEQ ID NO: 370 - DNA, 21 bp, Artificial
  PCR primer
agctccttgatctgctccttc

SEQ ID NO: 371 - DNA, 21 bp, Artificial
  PCR primer
gactcctgaggctgtgctatg

SEQ ID NO: 372 - DNA, 21 bp, Artificial
  PCR primer
gatccatgtttgggaaatcct

SEQ ID NO: 373 - DNA, 21 bp, Artificial
  PCR primer
tcctggtatgcaagatcaagg

SEQ ID NO: 374 - DNA, 21 bp, Artificial
  PCR primer
agagcgtcctttgagaactcc

SEQ ID NO: 375 - DNA, 21 bp, Artificial
  PCR primer
agcagctgaagcagaagtacg

SEQ ID NO: 376 - DNA, 21 bp, Artificial
  PCR primer
gctcatcagacaaaagcttgg